(19) United States
(12) Patent Application Publication
(10) Pub. No.: US 2015/0276709 A1
(43) Pub. Date: Oct. 1, 2015

(54) METHODS AND KIT FOR NUCLEIC ACID SEQUENCING

(71) Applicant: QuantuMDx Group Limited, Newcastle (GB)

(72) Inventors: Jonathan O'Halloran, Uckfield (GB); Christopher Adams, Newcastle (GB); Joseph H. Hedley, Tyne and Wear (GB); Sam Whitehouse, Newcastle Upon Tyne (GB)

(73) Assignee: QuantuMDx Group Limited, New Castle (GB)

(21) Appl. No.: 14/419,905

(22) PCT Filed: Aug. 5, 2013
(86) PCT No.: PCT/IB2013/002168

§ 371 Date: Feb. 5, 2015

Related U.S. Application Data
(60) Provisional application No. 61/680,212, filed on Aug. 6, 2012.

(51) Int. Cl. G01N 33/487; C12Q 1/68

(52) U.S. Cl. CPC G01N 33/48721; C12Q 1/6869; B82Y 15/00

(57) ABSTRACT

Various embodiments of the present disclosure generally relate to molecular biological protocols, equipment and reagents for the sequencing of long individual polynucleotide molecules.

### RELATED APPLICATIONS

The present invention claims priority to U.S. Provisional Application Ser. No. 61/680,212, filed Aug. 6, 2012, which is hereby incorporated by reference in its entirety.

### FIELD OF THE INVENTION

The present invention relates to molecular biological methods and sensor design, fabrication and use, for sequencing single nucleic acid (genomic DNA, RNA, cDNA, etc.) molecules and other molecules, to enable, for example, highly parallel, high throughput single molecule and long read length DNA sequencing and fragment length analysis.

### BACKGROUND OF THE INVENTION

DNA (deoxyribonucleic acid) is an often long polymer consisting of subunits called nucleotides. The chains of these single subunits form molecules called nucleic acids, of which DNA and RNA (ribonucleic acid) are by far the most commonly found examples in nature. Natural deoxyribonucleotides are comprised of one of four bases (adenine (A), cytosine (C), guanine (G) and thymine (T), along with a ribose/phoshpo backbone. In naturally occurring ribonucleotide populations Thymidine is replaced by Uracil (U). When polymerized through the formation of phosphodiester bonds at the 5′ and 3′ positions of the ribose backbone, nucleic acids may carry the genetic information in the cell. The bases in nucleic acids are able to form hydrogen bonds with one another, facilitating the formation of sable double-stranded molecules, each half of which is, in the case of DNA, a reverse complement of the other. DNA comprises two long chains of nucleotides comprising the four different nucleotides bases (e.g. AGTCATCGT . . . etc.) with a backbone of sugars and phosphate groups joined by ester bonds, twisted into a double helix and joined by hydrogen bonds between the complementary nucleotides (A hydrogen bonds to T and C to G in the opposite strand). The sequence of nucleotide bases along the backbone may harbor substantial amounts of information, and may comprise the vast majority of heritable information, such as individual hereditary characteristics.

The central dogma of molecular biology generally describes the normal flow of biological information as follows: DNA can be replicated to DNA, the genetic information in DNA can be ‘transcribed’ into RNA, such as messenger RNA (“mRNA”), and proteins can be translated from the information in mRNA. During translation, in a protein subunits (amino acids) are brought close enough to bond, in an order dictated by the sequence of the mRNA and ultimately, the DNA from which it was transcribed. This process involves the base-pairing of amino-acid adapter RNA molecules called tRNA (“transfer RNA”), each of which carries a specific amino acid dependent on its sequence to the mRNA sequence in the presence of a ribosome, which is itself a protein complex built around an rRNA (“ribosomal RNA”) core. Through this process, the genomic DNA sequence, using an mRNA intermediary and tRNA and rRNA constituents, specifies the sequence of amino acids to be assembled into polypeptides.

The term nucleic acid sequencing generally encompasses biochemical methods for determining the order of the nucleotide bases, adenine, guanine, cytosine, and thymine, in DNA or RNA molecules. The sequence of DNA constitutes the heritable genetic information in nuclei, plasmids, mitochondria, and chloroplasts that forms the basis for the developmental programs of living organisms. Genetic variations can cause disease, confer an increased risk of disease or confer beneficial traits. These variations can be inherited (passed on by parents) or acquired (developed as an adult, such as through a mistake in DNA replication). It is therefore of significant importance to know the sequence of these genetic molecules to gain a better understanding of life, molecular systems and disease.

DNA analysis was first widely celebrated with DNA Profiling (DNA Fingerprinting) and made commercially available in 1987, when a chemical company, Imperial Chemical Industries (ICI), started a blood-testing center in England. The technique was first reported by Sir Alec Jeffreys at the University of Leicester in England, and is now the basis of several national DNA databases, including the CODIS panel in the United states. The technique uses repetitive (“repeat”) sequences that are highly variable, called variable number tandem repeats (VNTRs), particularly short tandem repeats (STRs). VNTR loci are very similar between closely related humans, but so variable that unrelated individuals are extremely unlikely to have the same VNTRs. The amplification and subsequent fragment length analysis of the amplicons provides powerful genetic information about the identity or relatedness of individuals.

The advent of DNA sequencing has significantly accelerated biological research and discovery and expanded the use of DNA testing from simple profiles into disease diagnosis and even prediction. The rapid speed of sequencing attainable with modern DNA sequencing technology has been instrumental in the large-scale sequencing of the human genome, in the Human Genome Project. Related projects have generated the complete DNA sequences of many animal, plant, viral, and microbial genomes.

RNA sequencing, which for technical reasons is easier to perform than DNA sequencing, was one of the earliest forms of nucleotide sequencing. The major landmark of RNA sequencing, dating from the pre-recombinant DNA era, is the sequence of the first complete gene and then the complete genome of Bacteriophage MS2, identified and published by Walter Fiers and his coworkers at the University of Ghent (Ghent, Belgium), published between 1972 and 1976.

The chain-termination method developed by Frederick Sanger and co-workers in 1975 was the first method of DNA sequencing to be employed on a large scale. Prior to the development of rapid DNA sequencing methods in the early 1970s by Sanger in England and Walter Gilbert and Allan Maxam at Harvard, a number of laborious methods were used, such as wandering-spot analysis, as presented by Gilbert and Maxam in 1973, which reported the sequencing of 24 base-pairs

In 1976-1977, Allan Maxam and Walter Gilbert developed a DNA sequencing method based on chemical modification of DNA and subsequent cleavage at specific bases. The method requires radioactive or fluorescent labeling at one end of the DNA strand and purification of the DNA fragment to be sequenced Infrequent breaks are generated at one and sometimes two of the four nucleotide bases and this repeated in four reactions (G, A+G, C, C+T). This produces a series of labeled fragments, from the radiolabelled end to the first ‘cut’ site in each molecule and size-separated by gel electrophoresis, with the four reactions arranged side by side. Maxam-Gilbert sequencing was not readily taken up due to its technical complexity, extensive use of hazardous chemicals, and difficulties with scale-up. In addition, the method cannot easily be customized for use in a standard molecular biology kit.

The chain-termination or Sanger method requires a single-stranded DNA template, a DNA primer, a DNA polymerase, radioactively or fluorescently labeled nucleotides, and modified nucleotides, dideoxynucleotides triphosphates (ddNTPs) that terminate DNA strand elongation. The DNA sample is divided into four separate sequencing reactions, each containing the four standard deoxynucleotides (dATP, dGTP, dCTP and dTTP) and the DNA polymerase. To each of the four separate sequencing reactions is added only one of the four dideoxynucleotides (ddATP, ddGTP, ddCTP, or ddTTP). These dideoxynucleotides are the chain-terminating nucleotides, lacking the 3′-OH ribosyl group required for the formation of a phosphodiester bond between two nucleotides during DNA strand elongation. Incorporation of a dideoxynucleotide into the nascent (elongating) DNA strand therefore terminates DNA strand extension, resulting in various DNA fragments of varying length, each of which terminates at a site of integration of a dideoxy nucleotide. Thus if the identity of the dideoxyucleotide is known, the length of the fragments created will indicate the position in the sequence of the dideoxy base. The dideoxynucleotides are added at lower concentration than the standard deoxynucleotides to allow strand elongation sufficient for sequence analysis.

The newly synthesized and labeled DNA fragments are heat denatured, and separated by size (with a resolution of just one nucleotide) by gel electrophoresis on a denaturing polyacrylamide-urea gel. Each of the four DNA synthesis reactions is run in one of four individual lanes (lanes A, T, G, C); the DNA bands are then visualized by autoradiography or LTV light, and the DNA sequence can be directly read off the X-ray film or gel image. X-ray film was exposed to the gel, and when developed, the dark bands correspond to DNA fragments of different lengths. A dark band in a lane indicates a DNA fragment that is the result of chain termination after incorporation of a dideoxynucleotide (ddATP, ddGTP, ddCTP, or ddTTP). The terminal nucleotide base can be identified according to which dideoxynucleotide was added in the reaction giving that band. The relative positions of the different bands among the four lanes are then used to read (from bottom to top) the DNA sequence as indicated.

DNA fragments can be labeled by using a radioactive or fluorescent tag on the primer, in the new DNA strand with a labeled dNTP, or with a labeled ddNTP. There are some technical variations of chain-termination sequencing. In one method, the DNA fragments are tagged with nucleotides containing radioactive phosphorus for radiolabeling. Alternatively, a primer labeled at the 5′ end with a fluorescent dye is used for the tagging. Four separate reactions are still required, but DNA fragments with dye labels can be read using an optical system, facilitating faster and more economical analysis and automation. This approach is known as ‘dye-primer sequencing’. The later development by L Hood and co-workers of fluorescently labeled ddNTPs and primers set the stage for automated, high-throughput DNA sequencing.

The different chain-termination methods have greatly simplified the amount of work and planning needed for DNA sequencing. For example, the chain-termination-based “Sequenase” kit from USB Biochemicals contains most of the reagents needed for sequencing, prealiquoted and ready to use. Some sequencing problems can occur with the Sanger method, such as non-specific binding of the primer to the DNA, affecting accurate read-out of the DNA sequence. In addition, secondary structures within the DNA template, or contaminating RNA randomly priming at the DNA template can also affect the fidelity of the obtained sequence. Other contaminants affecting the reaction may consist of extraneous DNA or inhibitors of the DNA polymerase.

An alternative to primer labeling is labeling of the chain terminators, a method commonly called ‘dye-terminator sequencing’. One of major advantages of this method is that the sequencing can be performed in a single reaction, rather than four reactions as in the labeled-primer method. In dye-terminator sequencing, each of the four dideoxynucleotide chain terminators is labeled with a different fluorescent dye, each fluorescing at a different wavelength. This method is attractive because of its greater expediency and speed and is now the mainstay in automated sequencing with computer-controlled sequence analyzers (see below). Its potential limitations include dye effects due to differences in the incorporation of the dye-labeled chain terminators into the DNA fragment, resulting in unequal peak heights and shapes in the electronic DNA sequence trace chromatogram after capillary electrophoresis.

The analysis of nucleotide polymers (DNA and RNA) has become important in the clinical routine. However, cost and complexity remain major barriers to widespread global adoption. One reason for this is the complexity of the analysis requiring expensive devices that are able to sensitively measure up to four different fluorescence channels as experiments progress. Other reasons include the high cost of reagents, long and complex sample preparation steps and extensive computational power coupled with skilled bioinformaticians to assemble the resultant short-read sequences into clinically relevant constructs. The cheaper alternatives may require skilled technicians to run and interpret low-tech equipment, such as electrophoresis gels, but this too may be expensive and doesn't produce enough DNA data for high throughput whole genome sequencing applications.

### SUMMARY OF THE INVENTION

A new method of sequencing a plurality of polynucleotide molecules is disclosed in accordance with embodiments of the present invention. In some embodiments the method may be used to address issues of complexity, cost, time, and a requirement for long-read length and high through-put DNA Sequencing. Various embodiments used in connection with the present disclosure look to perform long read length, highly parallel, single molecule DNA sequencing in a cost effect device using a novel sequencing technique. In some embodiments of the technology the invention can be used for the analysis of DNA fragment lengths.

Some embodiments comprise a device for sequencing, or analyzing the length of a polynucleic acid molecule. In some aspects the device comprises a nanochannel with one dimension in the nm range. In some aspects an embodiment describes a channel having a width of less than 3 μm and a height of less than 100 nm. In some embodiments the channel is less that 50 nm in diameter. In yet more embodiments the channel diameter is less than 5 nm; and an array of nanostructure sensors, arrayed perpendicular or parallel to the nanochannel, having a sensitive assay region within said nanochannel such that a perturbation resulting from a passing fragment from a polynucleic acid molecule, or an individual base. In some embodiments each base will provide a unique electrical signature as it passes the nanostructure sensors either directly or through displacement of ions of a polynucleic acid passing through said sensitive assay region results in a specific signal being generated by said sensors. In some aspects the nanostructure sensor detects electrical charge. In some aspects the nanostructure detects a high-charge moiety. In some aspects the high charge moiety is a moiety of FIG. 7A-G or FIG. 8. In some aspects the nanostructure sensor detects buffer solution potential. In some aspects the nanostructure sensor detects fluorescence. In some aspects the nanostructure sensor detects buffer displacement. In some aspects the nanostructure sensor detects heat. In some aspects the nanostructure detects stress.

In some aspects the nanochannel is bounded by walls typically comprising one or more of Al2O3, SiN, Si, grapheme, polymetric materials, photoresist and SiO2. In some aspects the nanochannel is bounded by walls comprising at least one constituent not previously listed. In some aspects the nanochannel comprises a capping layer. In some aspects the nanostructure sensor comprises an array of nanowires, perpendicular or parallel to a nanochannel. In some aspects a nanostructure sensor comprises an array of carbon nanotubes perpendicular or parallel to a nanochannel. In some aspects the sensor comprises an array of graphene sheets, arrayed perpendicular or parallel to the nanochannel. In some aspects of this invention graphene sheets are orientated such that they stand up in the nanochannel providing the ability for single base differentiation. In some aspects the width of a sheet is 1 atom thick which in some embodiments can readily determine the nucleotide sequence at the single base resolution as the base to base distance is 3.4 angstroms. In some aspects the nanostructure sensors arrayed in the nanochannel comprise one or more individually addressed FET devices. In some aspects the nanostructure sensor detects electrical charge. In some aspects the nanostructure detects a high-charge moiety. In some aspects the high charge moiety is a moiety of FIG. 7A-G or FIG. 8. In some aspects the nanostructure sensor detects buffer solution potential. In some aspects the nanostructure sensor detects fluorescence. In some aspects the nanostructure sensor detects buffer displacement. In some aspects the nanostructure sensor detects heat. In some aspects the nanostructure detects stress. In some aspects the device comprises a plurality of said nanostructure sensors. In some aspects the device comprises a single nanostructure sensor. In some aspects the nanostructure sensors are positioned to detect perturbations of individual bases of a polynucleotide molecule passing by said sensors. In some aspects the nanostructure sensors operate in clusters of three. In some aspects the nanostructure sensors operate in clusters of two. In some aspects the nanostructure sensors operate individually. In some aspects the device comprise a transmitter that transmits said signal. In some aspects the nanochannel includes a solution and this solution may be a gel. In some aspects the solution conducts electricity. In some aspects the solution conducts an electric current that draws a polynucleic acid into or through said nanochannel. In some aspects the solution flows through said nanochannel. In some aspects the device comprises multiple nanochannels. In some aspects the device may be hand-held.

Some embodiments comprise a method of sequencing a single polynucleic acid molecule. In some aspects the method comprises providing an isolated polynucleic acid molecule in a solution; providing a nanostructure sensor having a sensitive assay region; drawing said isolated polynucleic acid past said sensitive assay region of said nanostructure sensor; and measuring a perturbation in said sensitive assay region, wherein said perturbation corresponds to an individual base of said isolated polynucleic acid molecule. In some aspects the perturbation is an electric charge in said sensitive assay region. In some aspects the perturbation is a volume displacement in said sensitive assay region. In some aspects the perturbation is fluorescence in said sensitive assay region. In some aspects the polynucleic acid molecule comprises a nucleotide-base specific modification. In some aspects the base-specific modification corresponds to a base-specific perturbation in said sensitive assay region. In some aspects the base-specific modification comprises base-specific addition of a molecule of FIG. 7A-G or FIG. 8. In some aspects the base-specific modification is incorporated into said polynucleic acid molecule during a template-directed nucleotide polymerization reaction. In some aspects the drawing said isolated polynucleic acid past said sensitive assay region of said nanostructure sensor comprises running a current or voltage through said solution. In some aspects the drawing said isolated polynucleic acid past said sensitive assay region of said nanostructure sensor comprises establishing a flow of said solution past said sensitive assay region. In some aspects the sensitive assay region is contained within a nanochannel. In some aspects the nanochannel has a width of less than 2.5 μm and a height of less than 70 nm. In some aspects the method comprises annealing a labeled probe to said isolated polynucleic acid molecule. In some aspects the labeled probe comprises DNA, RNA, peptide nucleic acid (PNA), morpholino, locked nucleic acid (LNA), glycol nucleic acid (GNA), threose nucleic acid (TNA), or a synthetic nucleotide polymer. In some aspects the labeled probe is a hexamer. In some aspects the labeled probe is a pentamer. In some aspects the labeled probe is a tetramer. In some aspects the labeled probe is end-labeled.

Some embodiments comprise a method of sequencing a target polynucleotide. In some aspects the method comprises: providing within an assay region an array of sensitive detection nanostructure sensors that generates a signal related to a property of an analyte that passes past the array within the assay region, wherein the assay region can be a nanofluidics channel; elongating the DNA or RNA molecule through the nanofluidics channel, such that the target polynucleotide passes within the sensitive nanostructure sensors operable field; detecting within the assay region a change in the signal that is characteristic of at least one nucleotide in the DNA or RNA polymer chain. In some aspects the method comprises continuous detection and measurements of the environment within the assay area, as the target DNA or RNA polymer moves through the assay region, thereby exposing each monomer in the polymer to the assay region one at a time. In some aspects the property is an electrical charge. In some aspects the property is fluorescence. In some aspects the property is heat. In some aspects the nanofluidics channel passes a protein past the sensitive nanostructure arrays. In some aspects the nanofluidics channel passes a metabolite past the sensitive nanostructure arrays. In some aspects the nanofluidics channel passes a gas through past the sensitive nanostructure arrays. In some aspects the nanofluidics channel passes metal ions through past the sensitive nanostructure arrays. In some aspects the reaction entity actively passes the DNA or RNA polynucleic acid polymer through the assay region. In some aspects the reaction entity passively passes the DNA or RNA polynucleic acid polymer through the assay region. In some aspects the reaction entity is a nanopore. In some aspects the reaction entity is a nanofluidic channel In some aspects reporter moieties are added to the nucleotides in DNA or RNA polymers prior to sequencing. In some aspects the nucleotide monomers carry a charge mass reporter moiety unique to that species of nucleotide (A, G, C & T). In some aspects the charge mass reporter is configured to be removable. In some aspects the charge mass reporter moiety is removed from the added nucleotide after detecting the signal, thereby allowing for the incorporation of the following nucleotide monomer. In some aspects the charge mass reporter moiety is configured not to affect polymerization of the nascent chain by the polymerase. In some aspects the charge mass reporter moiety is configured to protrude out from the nascent chain so as to be accessible to the assay region. In some aspects the added nucleotide further comprises a cleavable cap molecule at the 5′ phosphate group so that addition of another nucleotide is prevented until the cleavable cap is removed. In some aspects the linker is bound to the 5′ phosphate group of the added nucleotide, thereby acting as a cap. In some aspects the sensitive detection nanostructure is selected from the group consisting of a nanowire, a nanotube, a nanogap, a nanobead, a nanopore, a field effect transistor (FET)-type biosensor, a planar field effect transistor, a FinFET, a chemFET, an ISFET, Graphene based sensor, and any conducting nanostructures including, for example, nanostructures capable of sensing the perturbation in charge, fluorescence, stress, pressure, or heat. In some aspects the target polynucleotide and the primer comprise molecules selected from the group consisting of DNA, RNA, peptide nucleic acid (PNA), morpholino, locked nucleic acid (LNA), glycol nucleic acid (GNA), threose nucleic acid (TNA), synthetic nucleotide polymer, and derivatives thereof. In some aspects the added nucleotide monomer comprises a molecule selected from the group consisting of a deoxyribonucleotide, a ribonucleotide, a peptide nucleotide, a morpholino, a locked nucleotide, a glycol nucleotide, a threose nucleotide, a synthetic nucleotide, and derivatives thereof. In some aspects the means for detecting the signal are selected from the group consisting of piezoelectric detection, electrochemical detection, electromagnetic detection, photodetection, mechanical detection, acoustic detection and gravimetric detection.

Some embodiments comprise a device for sequencing a target polynucleotide. In some aspects the device comprises a microfluidics cassette comprising a sample reception element for introducing a biological sample comprising the target polynucleotide into the cassette; a lysis chamber for disrupting the biological sample to release a soluble fraction comprising nucleic acids and other molecules; a nucleic acid separation chamber for separating the nucleic acids from the other molecules in the soluble fraction; an amplification chamber for amplifying the target polynucleotide; an assay region comprising an array of one or more sensitive detection nanostructures that generate a signal related to a property of the nanostructures, wherein the assay region is configured to allow operable coupling of the target polynucleotide to the nanostructures; and a conducting element for conducting the signal to a detector. In some aspects the biological sample comprises any body fluid, cells and their extract, tissues and their extract, and any other biological sample comprising the target polynucleotide.

In some aspects the device is sized and configured to be handheld. In some aspects the device is sized and configured to fit into a mobile phone, smartphone, iPad, iPod, laptop computer, or other portable device. In some aspects the devices comprises at least 10 assay regions. In some aspects the devices comprises at least 100 assay regions. In some aspects the devices comprises at least 1000 assay regions. In some aspects the devices comprises at least 10,000 assay regions. In some aspects the devices comprises at least 100,000 assay regions. In some aspects the devices comprises 1,000,000 or over 1,000,000 assay regions. In some aspects the channel is incorporated using a Focused Ion beam. In some aspects the channel is fabricated using contact or non-contact photolithographic or shadow masking techniques. In some aspects the channel is fabricated using one or more of nanoimprinting, nanoembossing and nanostamping techniques. In some aspects the fabrication comprises electron beams, nanoinks or dip pen nano-lithographic tools, wet chemical etching, dry gaseous etching, thermal oxidation, chemical oxidation, ionic bombardment or a combination of two or more of said techniques. In some aspects multilayer planes are realized. In some aspects the layers are developed through selective milling, inclusion of sublimation chemistry and further layer deposition. In some aspects the nanowire or nanowires are parallel to the incoming fluid flow.

In some embodiments a method comprises: providing an array of sensitive detection nanostructure sensors, such as nanowire or nanotube FET sensors, that generate signals related to a property of a nanostructure. In some embodiments this array is within an assay region or housing. In some embodiments the nanostructure sensors are arrayed throughout a nanofluidic channel. The channel may have dimensions such that the polynucleotide such as DNA or RNA elongates through the channel. The sensors in the channel may be sensitive enough and able to measure the bases in a single molecule of a polynucleotide such as DNA or RNA as the molecule passes near the sensor. The nanostructure sensors may be geometrically spaced at various pitched distances to allow for the discrimination and identification of each base, or group of bases, or reporter moieties linked to one or more bases, or probes hybridized to the bases. In some embodiments this occurs as the elongated polynucleotide such as DNA or RNA flows, or is otherwise drawn across, through or made to pass through the channel, past the sensitive nanostructure sensors.

In some embodiment the sequencing device is a Nanochannel Nanowire Sequencing (NNS) Device. In some embodiments the sequencing device comprises at least one or more, up to an array of sensitive nanostructure sensors. These sensors may be operably coupled to a nanofluidic channel. In some embodiments sensing occurs when the polynucleotide such as DNA or RNA passes through the nanofluidic channel. In some embodiments the charges carried by the different nucleotides, or covalently added reporter groups, or hybridized oligo markers, within the polynucleotide such as DNA or RNA polynucleic acid polymer may be differentiated by the array of sensitive nanostructure sensors. In some embodiments base calling may be a function of the aggregation of data from each of the one or more sensors such as sensitive nanostructure sensors. In some embodiments the base calling may be calculated using an algorithm, thus allowing for base calling of the polynucleotide such as DNA or RNA sequence.

Some embodiments of the present disclosure describe novel biosensors, chemical reagents and synthetic nucleotides that can generally be utilized in such devices. Various embodiments used in connection with of the present disclosure describe a novel biosensor that comprises a sensitive nano-scale detection device. In some embodiments the device is capable at detecting electrical charges present at or near its surface (or charges of reporter moieties attached to the nucleotides), such as single nucleotides, or reporter moieties attached to single nucleotides within single strands of nucleic acid molecules, fed through a nanofluidic channel, which can be fabricated using numerous methodologies, as suggested in the examples. The sensitive detection device in turn monitors the changes in the environment (such as, but not limited to, changes in electric field, or changes in the potential of the buffer solution due to the presence or absence of certain molecules, such as nucleotides or nucleotide bases) at the sensors surface as the polynucleotide such as DNA or RNA passes by.

In some embodiments the sensors such as sensitive nanostructure sensors are capable of detecting the small changes in environment, such as changes caused by a polynucleotides such as a DNA or RNA molecule as it passes by. In some embodiments the sensors such as sensitive nanostructure sensors are capable of detecting the unique electrical signature of each base, or groups of bases. In some embodiments the sensor is a detector such as a nanowire, atomically thick graphene, or carbon nanotube FET device.

In some embodiments, the polynucleotide such as DNA or RNA can be comprised wholly or partially of synthetic nucleotide monomers. In some embodiments these synthetic monomers are different from naturally occurring polynucleotide constituents. In some embodiments each nucleotide carries a reporter moiety to increase the signal for the sensitive detection sensor. These synthetic nucleotides can, for example, comprise at least some standard nucleotides (or any modifications, or isoforms). These synthetic nucleotides may comprise one or more high negative charge mass reporter moieties. Each nucleotide base can carry a different high charge mass reporter moiety, thus allowing the sensitive nanostructure sensor (such as a nanowire, atomically thick graphene, or carbon nanotube FET sensor) to differentiate between each of the different nucleotide bases in the nucleotide polymer.

In some preferred embodiments of the method, the property of the detection method of the sensitive nanostructure sensor is an electrical charge.

In some preferred embodiments of the method, the property of the detection method of the sensitive nanostructure sensor is buffer displacement.

In some preferred embodiments of the method, the property of the sensitive nanostructure sensor is fluorescence.

In some preferred embodiments of the method, the property of the sensitive nanostructure sensor is heat of the reaction.

### BRIEF DESCRIPTION OF THE DRAWINGS

FIG. 1: An illustrative embodiment of a Nanochannel Nanowire Sequencing (NNS) device.

FIG. 2: A schematic of the processing needed to incorporate a nanochannel structure into a standard unwelled device.

FIG. 3: Steps in nanofabrication of the nanochannel structure.

FIG. 4: Sequencing reaction employing tagged oligonucleotide primer sequence and tagged dideoxynucleotides.

FIG. 5: Probe-based sequence-detection employing labeled hexamer probes.

FIG. 6: Amplification-based sequence detection employing labeled nucleotides.

FIG. 7A-G: Exemplary high-charge mass moieties used to label bases for charge-based detection.

FIG. 7A. An exemplary high-charge mass moiety.

FIG. 7B. An exemplary high-charge mass moiety.

FIG. 7C. An exemplary high-charge mass moiety.

FIG. 7D. An exemplary high-charge mass moiety.

FIG. 7E. An exemplary high-charge mass moiety.

FIG. 7F. An exemplary high-charge mass moiety.

FIG. 7G. An exemplary high-charge mass moiety.

FIG. 8: An exemplary high-charge linker and charged species.

FIG. 9A: Image of fabricated nanochannel viewed across the face of the device.

FIG. 9B: Image of the nanochannel of 9A viewed down the nanochannel groove.

FIG. 10: Image of fabricated nanochannel viewed across the face of the device.

FIG. 11: Vertical cross-sectional view of an exemplary nanochannel

FIG. 12: Vertical cross-sectional view of an exemplary nanochannel

FIG. 13: Image of an exemplary nanochannel.

FIG. 14A. Horizontal cross-sectional view of a nanochannel with nanowires indicated at top, middle and bottom with cross-marks.

FIG. 14b. Three successive vertical cross-sections of the three regions of the nanochannel in 14A. Cross sections correspond to the regions marked with the cross-marks.

FIG. 15: Image of Cy3 labeled DNA successfully drawn through a nanochannel.

FIG. 16 DNA translocating through a nanochannel in a controlled manner at approximately 5 um per second.

FIG. 17 Electrical read out of DNA translocating through a nanochannel.

### DETAILED DESCRIPTION

Aspects of the present disclosure describes a novel sequencing technology. Sequencing technology can be the general term used for determining the sequence of a single strand of a polynucleotide such as DNA or RNA molecule by either growing the nascent, reverse compliment, strand and detecting the addition of each new nucleotide in the growing polymer, or passing a double or single stranded DNA or RNA molecule through, on, or near a detection device, such that the sequence of nucleotides throughout the polynucleotide such as DNA or RNA polynucleic acid polymer can be detected. Using the more modern methods described above (methods employed by Helicos, 454 Life Sciences & Solexa), this can be performed by adding each separate nucleotide (adenine, guanine, cytosine or thymine) separately, in the presence of a polymerase and other elements required for polymerization, with a fluorescent reporter moiety ligated to the nucleotide and then observing the fluorescence using sensitive optical detection equipment. If there is fluorescence in the correct spectra for that nucleotide addition step, then the ‘base calling’ bioinformatics program may add the appropriate base in sequence. The reaction can then be washed and the next nucleotide in the cycle (wherein each of the four nucleotides Adenine, Guanine, cytosine and Thymine (or uracil for RNA) are added sequentially) can be added. This cycle is usually repeated until between approximately 25 bp to 900 bp or more (for example, depending on which method is used) worth of sequence data is obtained for each reaction. To enable whole genome sequencing, many thousands of these reactions can be performed in parallel.

Modern dye-terminator or chain-termination sequencing can produce a sequence that may have poor quality in the first 15-40 bases, a high quality region of 700-900 bases, and then quickly deteriorating quality. Automated DNA sequencing instruments (DNA sequencers) operating these methods can sequence up to 384 fluorescently labeled samples in a single batch (run) and perform as many as 24 runs a day. However, automated DNA sequencers may carry out only DNA-size-based separation (by capillary electrophoresis, the same technology used for DNA fragment length analysis for DNA profiling), detection and recording of dye fluorescence, and data output as fluorescent peak trace chromatograms. Sequencing reactions by thermocycling, clean-up and re-suspension in a buffer solution before loading onto the sequencer may be performed separately.

Over the past 5 years, so called NextGen sequencing technologies have emerged. Some of these are based on pyrosequencing, nanopore sequencing, reversible termination chemistry, etc. and these new high-throughput methods use methods that parallelize the sequencing process, producing thousands or millions of sequences at once.

As molecular detection methods are often not sensitive enough for single molecule sequencing (Helicos, Pacific Biosciences and Oxford Nanopore's methodologies are an exception), many approaches use an in vitro cloning step to generate many copies of each individual molecule. Emulsion PCR is one method, isolating individual DNA molecules along with primer-coated beads in aqueous bubbles within an oil phase. A polymerase chain reaction (PCR) then coats each bead with clonal copies of the isolated library molecule and these beads are subsequently immobilized for later sequencing. Emulsion PCR is used in the methods published by Marguilis et al. (commercialized by 454 Life Sciences, acquired by Roche), Shendure and Porreca et al. (also known as “polony sequencing”) and SOLiD sequencing, (developed by Agencourt and acquired by Applied Biosystems). Another method for in vitro clonal amplification is “bridge PCR”, where fragments are amplified upon primers attached to a solid surface, developed and used by Solexa (now owned by Illumina) These methods both produce many physically isolated locations which each contain many copies of a single fragment.

Once clonal DNA sequences are physically localized to separate positions on a surface, various sequencing approaches may be used to determine the DNA sequences of all locations, in parallel. “Sequencing by synthesis”, like the popular dye-termination electrophoretic sequencing, uses the process of DNA synthesis by DNA polymerase to identify the bases present in the complementary DNA molecule. Reversible terminator methods (used by Illumina and Helicos) use reversible versions of dye-terminators, adding one nucleotide at a time, detecting fluorescence corresponding to that position, then removing the blocking group to allow the polymerization of another nucleotide. Pyrosequencing (used by 454) also uses DNA polymerization to add nucleotides, adding one type of nucleotide at a time, then detecting and quantifying the number of nucleotides added to a given location through the light emitted by the release of attached pyrophosphates. “Sequencing by ligation” is another enzymatic method of sequencing, using a DNA ligase enzyme rather than polymerase to identify the target sequence. Used in the polony method and in the SOLiD technology offered by Applied Biosystems, this method uses a pool of all possible oligonucleotides of a fixed length, labeled according to the sequenced position. Oligonucleotides are annealed and ligated; the preferential ligation by DNA ligase for matching sequences results in a signal corresponding to the complementary sequence at that position.

Other methods of DNA sequencing may have advantages in terms of efficiency or accuracy. Like traditional dye-terminator sequencing, they are limited to sequencing single isolated DNA fragments. “Sequencing by hybridization” is a non-enzymatic method that uses a DNA microarray. In this method, a single pool of unknown DNA can be fluorescently labeled and hybridized to an array of known sequences. If the unknown DNA can hybridize strongly to a given spot on the array, causing it to “light up”, then that sequence is inferred to exist within the unknown DNA being sequenced. Mass spectrometry can also be used to sequence DNA molecules; conventional chain-termination reactions produce DNA molecules of different lengths and the length of these fragments can then be determined by the mass differences between them (rather than using gel separation).

These technologies are best known as ‘NextGen’ sequencing technologies. They rely on highly parallel sequencing of short fragments, sometimes sequencing the same base many times. The data from these short reads, anywhere from 25 bp-500 bp, are then assembled using bioinformatics that build the sequence fragments into a whole, using a scaffold sequencing (such as the published human genome) for guidance. This method finds it hard to resolve important structural elements and even other genotyping elements. It is not a technology for de novo assembly of genomes for which a scaffold does not exist due to these significant limitations. Furthermore, the clonal amplification step inherent to most of these technologies can introduce errors.

Therefore, for accurate de novo assemblies of genomes or other large DNA fragments, single molecule long read length sequencing is required.

There are new proposals for DNA sequencing, which are in development, but remain to be proven. These include labeling the DNA polymerase (Life Technologies ‘Starlight’ strategy, formerly, Visigen), reading the sequence as a DNA strand, or strands, or a DNA strand with markers hybridized or linked to the DNA, translocates through nanopores, or using nano-edge probe arrays that are stepped with sub-Angstrom resolution over a stretched and immobilized ssDNA (Reveo), a technique that uses single-photon detection, fluorescent labeling and DNA electrophoresis with detection using plasmonic nanostructures (base4innovation), and microscopy-based techniques, such as AFM or electron microscopy that are used to identify the positions of individual nucleotides within long DNA fragments by nucleotide labeling with heavier elements (e.g., halogens) for visual detection and recording.

Helicos, Pacific Biosciences and Oxford Nanopore have developed technologies that sequence single molecules, therefore they do not require this step. The single-molecule method developed in the Quake laboratory (later commercialized by Helicos) skips this amplification step, directly fixing DNA molecules to a surface. The Nanopore methodologies that are being commercialized by Oxford Nanopore, Genia, Nabsys and others, sense nucleotides, or groups of nucleotides as they translocate through a nanopore. Pacific Bioscience have developed Zero Mode Wavelength devices and a method for immobilizing a single polymerase within them, thereby allowing the detection of fluorescence emitted from the polymerization reaction from a single polymerase.

With exception of methods using mass spec, nanopores and microscopy-based techniques, several methods presently available, or in development generally require the use of expensive optical equipment and complex software. Furthermore, mass spec, and microscopy-based techniques may require bulky equipment that may limit their deployment and certainly can drive costs up.

The sequencing of the human genome and the subsequent studies have since demonstrated the great value in knowing the sequence of a person's DNA. The information obtained by genomic DNA sequence analysis can provide information about an individual's relative risk of developing certain diseases (such as breast cancer and the BRCA 1&2 genes). Furthermore, the analysis of DNA from tumors can provide information about stage and grading. To date however, we have been unable to resolve much of the structural variation in the human genome, due to the short reads of present Next Generation DNA Sequencing technologies, as described above, can only resolve short stretches of sequence and are therefore unsuitable to resolve large scale structural variation. Thus much of the genomic variation remains unresolved.

Infectious diseases, such as those caused by viruses or bacteria also carry their genetic information in nucleotide polymer genomes (either DNA or RNA). Many of these have now been sequenced, (or enough of their genome sequenced to allow for a diagnostic, or drug susceptibility test to be produced) and the analysis of infectious disease genomes from clinical samples (a field called molecular diagnostics) has become one of important methods of sensitively and specifically diagnosing disease.

Measurements of the presence or absence, as well as the abundance of mRNA species in samples can provide information about the health status of individuals, the disease stage, prognosis and pharmacogenetic and pharmacogenomic information. These expression arrays are fast becoming tools in the fight against complex disease and may gain in popularity as prices begin to fall.

In some embodiments, the present direct sequencing methods and components can detect the individual bases within a polynucleotide such as a DNA or RNA molecule as it passes past a sensitive nanostructure sensor due to the action of flow, or other method of moving an elongated, linearly extended, uncoiled or straightened DNA or RNA molecule through a nanofluidic channel which feeds the DNA over, near or past the array of sensitive nanostructure sensors such that the individual nucleotide bases within the DNA or RNA are sufficiently close to cause a change in properties, unique to each base, or group of bases, in the array of sensitive nanostructure sensors. The arrayed sensitive nanostructure sensors (such as nanowire, atomically thick graphene or nanotube FET sensors) detect the charge of each nucleotide base, our groups of nucleotide bases and these changes in property (such as conductance) of the sensitive nanostructure sensors as the polynucleotide such as DNA or RNA passes over them, can be used to resolve the base sequence of the polymer, in singular and in combination with all the sensitive nanostructure sensors in the array.

In other methods using the Nanowire Nanochannel Sequencer (NNS) device, the incorporation of synthetic nucleotides or synthetic bases that carry a reporter (such as a ‘high charge-mass’ reporter moiety covalently or other, linked to the nucleotide) into the DNA or RNA polymer, via PCR or other method, that carry a reporter moiety that cause a larger change in properties of the sensitive nanostructure sensor than natural nucleotides themselves. These nucleotides can be incorporated into the DNA or RNA polynucleic acid polymer via PCR or another method. They can be added as single nucleotides, such as cytosine, such that all cytosines within the DNA or RNA polynucleic acid polymer carry a synthetic reporter moiety. This can then be repeated for each of the other nucleotides. The reporter moiety or moieties may be added during polynucleotide synthesis or added via modification to a preexisting polynucleotide. Each of the groups can then be sequenced in the NNS device and the bioinformatics can build up the sequence reads by calculating the position of each of the four different reporter moieties and speed of flow of the DNA or RNA as it passes through the nanofluidic channel. In another method, all four synthetic nucleotides could be incorporated into a single channel and the reporters thus act to amplify the signal from each of the nucleotides in the DNA or RNA polymer.

In yet further methods for using the NNS device, an altered Sanger dye terminator sequencing approach can be used. In this methodology the primer for each sequencing run will be covalently, or otherwise, linked to a unique reporter moiety. Furthermore, in the reaction mix, terminating nucleotides with a reporter moiety unique to each of the four nucleotides, can be covalently or other, linked to it. As in a standard Sanger sequencing PCR reaction, the terminating nucleotides are at a concentration such that long reads are attainable. The plurality of different sequence fragments are fed through the NNS device and the bioinformatics determines the terminating base, relative to the primer reporter moiety and the speed of flow through the nanochannel. Therefore a sequence associated with each unique primer can be built up. As millions of NNS devices can be arrayed on a single chip, this provides the ability to perform massively parallel Sanger sequencing. Furthermore, due to the unique signature of the primer reporter moieties, this sequencing method can perform multiple sequences in a single reaction (limited only by the number of unique reporter moieties that are available, or can be developed).

The sensitive nanostructure sensor can be a nanowire FET sensor and can be created using standard CMOS (Complementary metal-oxide semiconductor) processing, or other fabrication methodologies well known to those familiar with the art such as those involving photolithography, shadow masking, electron beam lithography, nanoprinting, embossing, moulding, polishing, etching, oxidation, doping, deposition including chemical (or chemically enhanced), sputtering, evaporative deposition and structure growth. In some embodiments the sensors can be single sensors; in other embodiments the arrayed in arrays of more than at least two. In other embodiments they can be arrayed in hundreds. In yet more embodiments they can be arrayed in thousands. In further embodiments they can be arrayed in millions. In other embodiments they can be arrayed in billions or more.

As used herein in some aspects of embodiments, a “sensitive detection nanostructure” can generally be any structure (nanoscale or not) capable of generating a signal in response to a change in a property of the nanostructure within an assay region. As used herein an “assay region” refers generally to the area or region in which the nanostructure or nanostructures at least partially reside, and cause the DNA or RNA to be just in close enough physical proximity to exhibit a change in property and generate a signal in response to the different nucleotides within the DNA or RNA polynucleic acid polymer as they pass over, through, under or in the sensitive nanostructure. In preferred embodiments, such a change in property may be caused by a change in charge, or potential across a buffer due, to a charged molecule (such as a nucleotide in a DNA or RNA polymer) within the assay region or due to buffer displacement. Typically, the nanostructure is sensitive to changes at or near its surface (such as with nanowire or carbon nanotube FET biosensors), or as molecules pass through it (such as nanopore biosensors) although the assay region may extend beyond the surface of the nanostructure to include the entire region within the field of sensitivity of the nanostructure. The nanostructure is preferably also coupled to a detector that is configured to measure the signal and provide an output related to the measured signal. At any point along the length of the nanostructure, it may have at least one cross-sectional dimension less than about 500 nanometers, typically less than about 200 nanometers, more typically less than about 150 nanometers, still more typically less than about 100 nanometers, still more typically less than about 50 nanometers, even more typically less than about 20 nanometers, still more typically less than about 10 nanometers, and even less than about 5 nanometers. In other embodiments, at least one of the cross-sectional dimensions can be less than about 2 nanometers, or about 1 nanometer. In one set of embodiments the sensitive detection nanostructure can be at least one cross-sectional dimension ranging from about 0.5 nanometers to about 200 nanometers.

As used in various embodiments, a nanowire is an elongated nanoscale semiconductor which, at any point along its length, has at least one cross-sectional dimension and, in some embodiments, two orthogonal cross-sectional dimensions less than 500 nanometers, preferably less than 200 nanometers, more preferably less than 150 nanometers, still more preferably less than 100 nanometers, even more preferably less than 70, still more preferably less than 50 nanometers, even more preferably less than 20 nanometers, still more preferably less than 10 nanometers, and even less than 5 nanometers. In other embodiments, the cross-sectional dimension can be less than 2 nanometers or 1 nanometer. In one set of embodiments the nanowire has at least one cross-sectional dimension ranging from 0.5 nanometers to 200 nanometers. Where nanowires are described having a core and an outer region, the above dimensions relate to those of the core. The cross-section of the elongated semiconductor may have any arbitrary shape, including, but not limited to, circular, square, rectangular, elliptical, tubular, fractal or dendritic. Regular and irregular shapes are included. A non-limiting list of examples of materials from which nanowires of the invention may be made appears below. Nanotubes are a class of nanowires that find use in the invention and, in one embodiment, devices of the invention include wires of scale commensurate with nanotubes. As used herein, a “nanotube” is a nanowire that has a hollow core or core material differential to that of the nanowire and includes those nanotubes know to those of ordinary skill in the art. A “non-nanotube nanowire” is any nanowire that is not a nanotube, such as a Graphene sheet. In one set of embodiments of the invention, a non-nanotube nanowire having an unmodified surface (not including an auxiliary reaction entity not inherent in the nanotube in the environment in which it is positioned) is used in any arrangement of the invention described herein in which a nanowire or nanotube can be used. A “wire” refers to any material having conductivity at least that of a semiconductor or metal. For example, the term “electrically conductive” or a “conductor” or an “electrical conductor” when used with reference to a “conducting” wire or a nanowire refers to the ability of that wire to pass charge through itself. Preferred electrically conductive materials have a resistivity lower than about 10⁻³, more preferably lower than about 10⁻⁴, and most preferably lower than about 10⁻⁶ or 10⁻⁷ ohmmeters.

A Nanopore generally has one or more small holes in an electrically isolated or insulating membrane. A Nanopore is generally, but not limited to a spherical structure in a nanoscale size with one or more pores therein. According to some aspects, a nanopore is derived from carbon or any conducting material.

A Nanobead is generally a spherical structure in a nanoscale size. The shape of nanobead is generally spherical but can also be circular, square, rectangular, elliptical and tubular. Regular and irregular shapes are included. In some examples, the nanobead may have a pore inside.

A Nanochannel is generally a channel with one dimension in a nanometer or nanoscale size. The shape of nanochannel is generally elongated and straight, but can also take on any other form factor, as long as the dimensions of the height and width are in the nano scale. Regular and irregular shapes are included and dependent upon fabrication methodology employed and include examples where then length of the channel from any start point to end point is greater than the vector distance between said points.

A Nanogap is generally used in a biosensor that consists of separation between two contacts in the nanometer range. It senses when a target molecule, or a number of target molecules hybridize or binds between the two contacts allowing for the electrical signal to be transmitted through the molecules.

A sequence (noun) is the identity and order of nucleic acid bases in a polynucleic acid. To sequence (verb) is to determine the identity and order of nucleic acid bases in a polynucleic acid.

A sensitive assay region is a region within which a sensor such as a nanosensor can detect a permutation in a sensed attribute or characteristic that can be correlated with the identity of an individual base in a polynucleic acid.

A perturbation is any change in a sensed attribute or characteristic, such as a change within a sensitive assay region.

A transmitter is a device that conducts or transmits information from a sensor, such as a detected perturbation, to a receiving device which may be outside of an NNS.

A specific signal is a signal generated by a sensor in response to a perturbation that can be uniquely correlated with the presence of a base of known identity in a sensitive assay region.

A solution is a liquid in which a polynucleic acid is soluble and having a viscosity compatible with flow through an NNS. In some embodiments herein the solution conducts electricity.

Height, as defined herein, is the smallest cross-sectional measurement in a nanochannel.

Width, as defined herein, is the second smallest cross-sectional measurement in a nanochannel, and is measured perpendicular or nearly perpendicular to the nanochannel height.

The foregoing nanostructures, namely, nanowire, nanotube, nanopore, nanobead, and nanogap are described to provide the instant illustration of some embodiments, and not to limit the scope of the present invention. In addition to the foregoing examples, any nanostructure that has a nanoscale size and is suitable to be applied to nucleic acid sequencing methods and apparatus as disclosed in the application should also be considered to be included in the scope of the invention.

### The Sensors

In general, nucleotide sequencing strategies for use with nanostructures or nanosensors sense the charge at, or near the surfaces, or across a nanogap or nanopore, which cause a measurable change in their properties (such as field effect transistors, nanogaps, or piezoelectric nanosensors). The charge sensed by the nanostructure can be directly originated from the nucleotide within the DNA or RNA polymer. In some embodiments, one or all of the nucleotides within a DNA or RNA polynucleic acid polymer are linked to a high charge mass reporter moiety, which are described in detail elsewhere in the specification.

In some embodiments the sensors are nanostructure sensors, such as nanowire, atomically thick graphene or nanotube FET sensors, that generate signals related to a property of a nanostructure. In some embodiments the nanostructure sensors are arrayed throughout a nanofluidic channel. The channel may have dimensions such that the polynucleotide such as DNA or RNA elongates through the channel. The sensors in the channel may be sensitive enough and able to measure the bases in a single molecule of a polynucleotide such as DNA or RNA as the molecule passes near the sensor. The nanostructure sensors may be geometrically spaced at various pitched distances to allow for the discrimination and identification of each base, or group of bases, or reporter moieties linked to one or more bases, or probes hybridized to the bases. In some embodiments this occurs as the elongated polynucleotide such as DNA or RNA flows, or is otherwise drawn across, through or made to pass through the channel, past the sensitive nanostructure sensors.

In some embodiments the sensors such as sensitive nanostructure sensors are capable of detecting the small changes in environment, as the polynucleotide such as DNA or RNA passes by a detector such as a nanowire, or carbon nanotube FET device.

In some preferred embodiments of the method, the property of the detection method of the sensitive nanostructure sensor is an electrical charge, fluorescence, heat of the reaction, conductance of the sample or of the contents of a nanochannel.

Field effect generally refers to an experimentally observable effect symbolized by F (on reaction rates, etc.) of intramolecular columbic interaction between the center of interest and a remote unipole or dipole, by direct action through space rather than through bonds. The magnitude of the field effect (or ‘direct effect’) may depend on the unipolar charge/dipole moment, orientation of dipole, shortest distance between the center of interest and the remote unipole or dipole, and on the effective dielectric constant. This is exploited in transistors for computers and more recently in DNA field-effect transistors used as nanosensors.

A Field-effect transistor (FET) is generally a transistor, which may use the field-effect due to the partial charges of biomolecules to function as a biosensor. The structure of FETs can be similar to that of metal-oxide-semiconductor field-effect transistor (MOSFETs) with the exception of the gate structure which, in biosensor FETs, may be replaced by a layer of immobilized probe molecules which act as surface receptors.

In some embodiments the sensors detect one or more of the signals selected from the group consisting of piezoelectric signals, electrochemical signals, electromagnetic signals, photon signals, mechanical signals, acoustic signals, heat signals and gravimetric signals.

### The Substrate—Preparation and Detection

In some embodiments, the direct sequencing may begin by simply feeding, or flowing, or otherwise causing or allowing the transport of a single polynucleic acid molecule such as a DNA or RNA polynucleic acid polymer over, past or through the sensitive nanostructure sensor; each nucleotide changes the sensor properties differently to the others, thus the sensor is able to detect sequence of nucleotides in the DNA/RNA polymer.

In some embodiments the length of a fragment of DNA, RNA, protein or other molecular can be determined by elongating the molecules through and translocating it through the nanochannel. As the front of the molecule enters the sensing region of the nanostructure sensor in the nanochannel a signal is generated. This signal stops when the end of the translocating molecule exits the sensing region of the nanostructure sensor. By having two or more nanostructure sensors in the nanochannel the speed of translocation can be determined and therefore the length of the molecule (DNA has a base to base distance of 3.4 Angstroms).

In some embodiments, the substrate may be an elongating polynucleic acid sequence that enters a nanostructure as it is being synthesized. In some embodiments the nucleic acid is single-stranded. In some embodiments the nucleic acid is double stranded. In some embodiments the nucleic acid comprises both a substrate and annealed labeled probes of known sequence.

In some embodiments, the sequencing reaction may begin by the inclusion of probes of known sequence that specifically hybridize to complimentary sequencing on the polynucleic acid such as the DNA or RNA polymer. The polynucleic acid such as the DNA or RNA, with which these hybridized probes can then be fed, flowed, or otherwise made to pass through the nanochannel and the array of sensitive nanostructure sensors can detect their positions and with information about the flow speed, computationally resolve their position. By repeating this for multiple probes that cover all sequence combinations, the method can resolve the sequence of an entire polynucleotide fragment up to and including a full length chromosome fed, flowed or otherwise made to pass, through the nanochannel. In some embodiments, the probes can have unique reporter moieties linked to them, such that all, or some, probes can be run in the same reactions, in multiplex.

These probes (short nucleic acid molecules, often referred to as oligonucleotides) can generally be a single stranded nucleotide polymer molecule, ssDNA, RNA, PNA, Morpholino, or other synthetic nucleotide. Furthermore, the ‘probe’ sequence can generally be reverse complimentary to the ‘target’ nucleic acid molecule to be sequenced and sufficiently long to facilitate hybridization. Generally the probe length will be 6 base pairs. In some methods the probe sequence can be 5 base pairs and in other methods the probes are 4, 3 or 2 base pairs. In yet more variations of the method, the probe sequence can be 7, 8, 9 or 10 base pairs. In further methods the probe length can be between 11-100 base pairs.

The probes preferably comprise molecules selected from the group consisting of DNA, RNA, peptide nucleic acid (PNA), morpholino, locked nucleic acid (LNA), glycol nucleic acid (GNA), threose nucleic acid (TNA), synthetic nucleotide polymer, and derivatives thereof.

In some embodiments, short adaptamers (another short oligonucleotide of known sequence) can generally be ligated to the target polynucleotide. This enables bar coding of different sequences, such as criminal or clinical sequences, such that one may run many different samples at once. In this method, each adaptamer will have a unique reporter moiety attached to it to enable its associated sequence to be distinguishable from the others.

In some embodiments coded or labeled PCR primers can be used to create a plurality of amplicons that can be analyzed in the NNS device. The analysis can comprise direct sequencing of the base pairs within each amplicons. The analysis can comprise analysis of amplicon lengths.

In various embodiments, labeled nucleotides may be incorporated into the polynucleic acid such as DNA or RNA polymers prior to introduction to the NNS device. These polynucleotides such as DNA or RNA polymers are detected as they pass the nanostructure sensor. In some embodiments, these nucleotides can be natural nucleotides. In some embodiments, the nucleotides are synthetic and comprise one or more of nucleotides, Adenine, Guanine, Cytosine and Thymine, plus isoforms of these bases (such as Inosine) with a reporter moiety attached, for instance, at the C5 position of pyrimidines or the C7 of the purines

In some embodiments of the present disclosure describes the use of synthetic nucleotides covalently linked to a highly charged reporter molecule amplifies the signal of the translocating molecule, or bases within the molecule. The reporter moiety can be varied for each nucleotide in order to carry a differing charge allowing the sensitive detection nanostructure to discriminate between nucleotides based on charge.

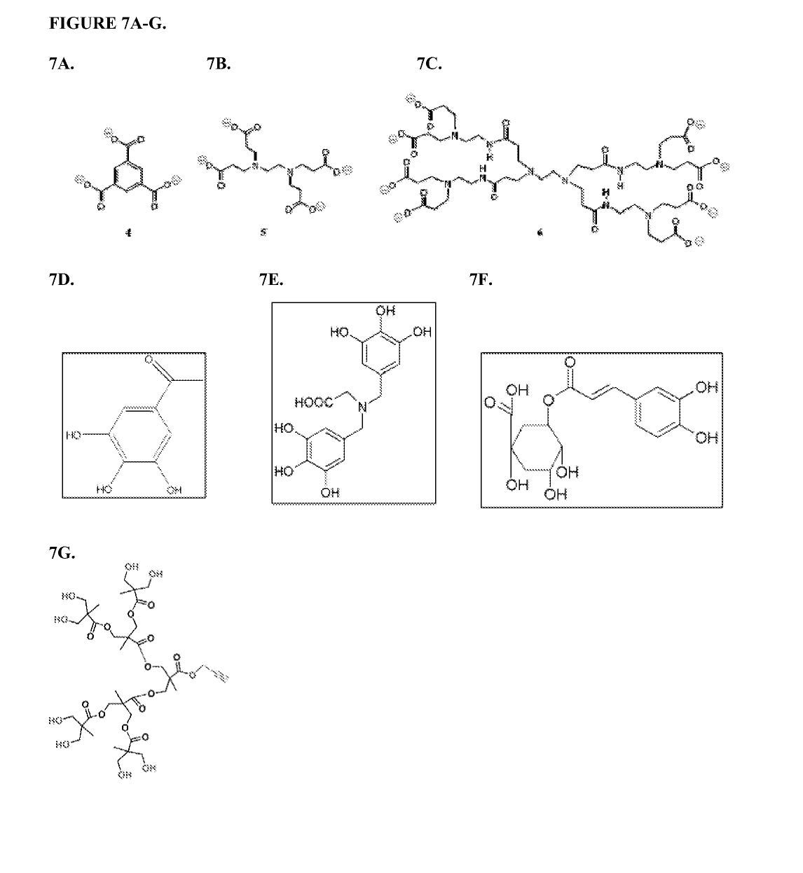

In some embodiments, the high charge mass moiety comprises but is not limited to, an aromatic and/or aliphatic skeleton comprising one or more of an amino group, an alkyne, an azide, an alcohol hydroxyl group, a phenolic hydroxy group, a carboxyl group, a thiol group or a charged metal species, or paramagnetic species or magnetic species or any combinations thereof. The high charge mass moiety may comprise one or more of the groups depicted in FIG. 7A-G, or derivatives thereof. High charge moieties are further discussed in U.S. Patent Application Publication No. 2011/0165572 A1, published Jul. 7, 2011, which is hereby incorporated by reference in its entirety, in U.S. Patent Application Publication No. 2011/0294685 A1, published Dec. 1, 2011, which is hereby incorporated by reference in its entirety, and in U.S. Patent Application No. 2011/0165563 A1, published Jul. 7, 2011, which is hereby incorporated by reference in its entirety. In some embodiments the nucleotides are labeled with one or more of the labels in FIGS. 7A through 7G. For example, in some embodiments the nucleotide A is unlabeled, T is labeled with the moiety in 7A, G is labeled with the moiety is 7B, and C is labeled with the moiety in 7C. Alternately, G may be unlabeled, C may be labeled with the moiety in 7D, A may be labeled with the moiety in 7E, and T may be labeled with the moiety in 7F. The moiety which labels each nucleotide is not constrained, provided that three of the four nucleotides are labeled such that all four bases, when passing through a nanochannel, each has a distinct measurable signal.

In some embodiments the base-specific reporter moiety is a fluorophore. A number of fluorophores that can be used to tag specific nucleotide populations are known in the art. A number of fluorophores are commercially available, for example from MoBiTec GmbH, Germany or Life Technologies. Some fluorophores include 2′-(or-3′)-O-(N-methylanthraniloyl) NTP, 2′-(or-3′)-O-(trinitrophenyl) NTP, BODIPY® FL 2′-(or-3′)-O-(N-(2-aminoethyl)urethane) NTP, Alexa Fluor® 488 8-(6-aminohexyl)amino NTP, or ATTO 425, ATTO 488, ATTO 495, ATTO 532, ATTO 552, ATTO 565, ATTO 590, ATTO 620, ATT0655, ATTO 680. In each ATTO dye, the numerical suffix indicates the absorbance spectrum. Thus an number of fluorescent dyes can be employed such that each base is labeled with a specific dye.

In some embodiments the base-specific reporter moiety is a FRET, with the donor or acceptor being immobilized on the nanostructure sensors. Different FRET molecules can be associated with each of the four bases.

In certain embodiments of the method, a base may incorporate a linker. Exemplary linkers include nucleotide modifications such as N⁶-(6-Amino)hexyl-, 8-[(6-Amino)hexyl]-amino-, EDA (ethan-diamine), Aminoallyl-, and 5-Propargylamino-linkers.

A linker may comprise a molecule of the following general formula:

R-Lₓ-R

Wherein, L comprises a linear or branched chain comprising of but not limited by an alkyl group, an oxy alkyl group, hydrocarbon, a hydrazone, a peptide linker, or a combination thereof, and R may comprise a nucleotide or nucleoside or polynucleic acid, or a label linked thereto.

In some embodiments, L may comprise a linear chain. The length of this chain is comprised of but not limited to 1-1800 repeat units. That is, the chain may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 850, 900, 950, 1,000, 1,100, 1,200, 1,300, 1,400, 1,500, 1,600, 1,700, or 1,800 repeat units.

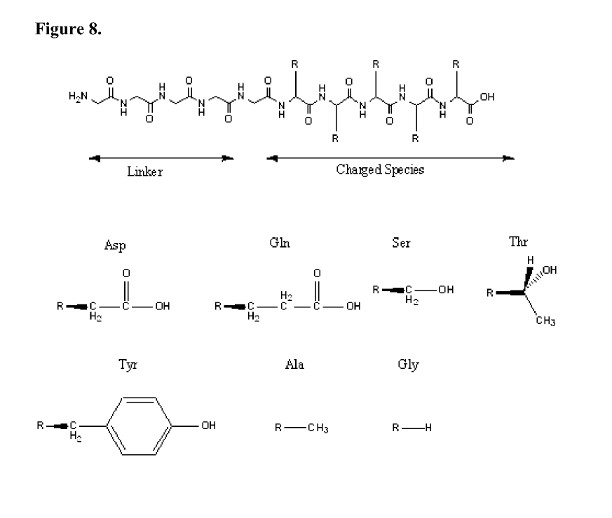

In some embodiments the charged species or the fluorophore may be intergrated into the linker by incorporating certain charged species along the chain. An example of such is given in FIG. 8, using, but not limited to, amino acid repeat units that incorporate R groups indicated therein that can carry charge that can affect the FET device. These species may also be able to act as a chelating group to bind other species such as magnetic or paramagnetic ions or particles.

In some embodiments a sequencing by synthesis reaction can be performed in the nanochannel, with the DNA or RNA molecule to be sequenced captured (by an electrical field, or tethered) in the nanochannel and sequencing buffer, dNTPs and polymerase flowed into the channel. The nucleotide incorporated into the DNA polymer prior to adding to the NNS device may also comprise a cleavable cap molecule so that addition of another nucleotide is prevented until the cleavable cap is removed, such as an ester. In some other embodiments, the linker can be bound to the nucleotide, thereby acting as a cap. A partial list of capped NTPs include 5-(3-Amino-1-propynyl)-2′-, and 7-(3-Amino-1-propynyl)-7-deaza-2′-NTP modifications. A review of cleavable fluorescent nucleotides is provided in Turcatti et al, Nucleic Acids Res. 2008 March; 36(4): e25, published online Feb. 7, 2008, which is hereby incorporated by reference in its entirety.

The target polynucleotide preferably comprise molecules selected from the group consisting of DNA, RNA, peptide nucleic acid (PNA), morpholino, locked nucleic acid (LNA), glycol nucleic acid (GNA), threose nucleic acid (TNA), synthetic nucleotide polymer, and derivatives thereof. The added nucleotide preferably comprises a molecule selected from the group consisting of a deoxyribonucleotide, a ribonucleotide, a peptide nucleotide, a morpholino, a locked nucleotide, a glycol nucleotide, a threose nucleotide, a synthetic nucleotide, and derivatives thereof.

The substrate may be drawn or forced through the nanochannel. A number of approaches to drawing the substrate through a nanochannel are contemplated. A polynucleotide may be drawn through a nanochannel by a flowing fluid passing through the nanochannel, by a pressure flux driving the fluid through the nanochannel, by an electromagnetic force such as a positive change, by gravity or other means.

### The Detection of the Substrate

In some embodiments, the sensitive detection nanostructure is selected from the group consisting of a nanowire, a nanotube, a nanogap, a nanobead, a nanopore, a field effect transistor (FET)-type biosensor, a planar field effect transistor, atomically thick graphene, graphene transistor and any conducting nanostructures.

In some embodiments, the signal detected is selected from the group consisting of piezoelectric detection, electrochemical detection, electromagnetic detection, photon detection, mechanical detection, acoustic detection, heat detection, gravimetric detection, and displacement of sample buffer in the nanochannel.

### The Apparatus—Additional Features

An apparatus for sequencing a target polynucleotide is disclosed in accordance with other embodiments of the present invention. The apparatus may comprise: an assay region comprising a sensitive detection nanostructure sensor capable of generating a signal caused by changes on and near the surface of the nanostructure (such as electrical field, or a fluorescence, etc.), and a nanochannel, that acts as a means to bring nucleotide polymers close enough to the sensitive detection nanostructure sensor such that each nucleotide in the polymer causes a change on or near the surface (such as an electrical field) of the sensitive detection nanostructure sensor, as it passes the sensor. In some embodiments, the apparatus may further comprise a pico-well or a microfluidics channel, or flow cell arrayed with the sensitive detection nanostructure sensors, wherein the biological sample comprises any body fluid, cells and their extract, tissues and their extract, and any other biological sample comprising nucleotides, extracted DNA, PCR (or other amplification methodologies, such as LAMP, RPA and other isothermal methods) amplified samples, synthesized oligos, or any other sample containing nucleotide polymers.

In some embodiments, the apparatus may comprise a microfluidics cassette. The microfluidics cassette may comprise a sample reception element for introducing a biological sample comprising the target polynucleotide into the cassette; a lysis chamber for disrupting the biological sample to release a soluble fraction comprising nucleic acids and other molecules; a nucleic acid separation chamber for separating the nucleic acids from the other molecules in the soluble fraction; an amplification chamber for amplifying the target polynucleotide; an assay region comprising an array of one or more NNS devices. In some examples, the apparatus can be used for the biological or clinical sample, which can be any body fluid, cells and their extract, tissues and their extract, and any other biological or clinical sample comprising the target polynucleotide. The apparatus for sequencing disclosed in some embodiments herein can be is sized and configured to be handheld, low through-put benchtop (for clinical applications), or in high throughput.

### The Sample Sources

In some embodiments, samples are extracted using methods known in the art for nucleic acid extraction. In some embodiments samples are solubilized or lysed prior to sequencing analysis. In some embodiments raw samples may be run in the apparatus, such that the sensor requires no pre-processing, such as lysis, extraction, PCR, etc., of the sample and can sequence DNA free within unextracted samples. In some embodiments samples are extracted and polynucleotides are labeled as contemplated herein.

Samples contemplated herein include but are not limited to, blood, urine, general crime scene material, semen, environmental samples, wastewater, ocean water, fresh water, plant material, dissolved tissue, and other sample matrices.

### The Nanochannels

In some embodiments a sample comprising a polynucleotide to be sequenced is channeled, run or elongated through a nanochannel, such as a nanochannel on a nanofabricated chip. Nanochannels consistent with the disclosure herein may be cross-sectionally rectangular, square, elliptical, semi-elliptical, circular, semi-circular, triangular, trapezoid, polygon or v-shaped, and may have sharp corners or round edges. Wells may be open-topped or may be enclosed in the nanofabrication chip.

Nanochannels may be about 2 μm across at their widest points. Alternately, wells may be less than 0.1 μm, 0.1 μm, 0.2 μm, 0.3 μm, 0.4 μm, 0.5 μm, 0.6 μm, 0.7 μm, 0.8 μm, 0.9 μm, 1.0 μm, 1.1 μm, 1.2 μm, 1.3 μm, 1.4 μm, 1.5 μm, 1.6 μm, 1.7 μm, 1.8 μm, 1.9 μm, 2.0 μm, 2.1 μm, 2.2 μm, 2.3 μm, 2.4 μm, 2.5 μm, 2.6 μm, 2.7 2.8 μm, 2.9 μm, 3.0 μm, 3.1 μm, 3.2 μm, 3.3 μm, 3.4 μm, 3.5 μm, 3.6 μm, 3.7 μm, 3.8 μm, 3.9 μm, 4.0 μm, 4.1 μm, 4.2 μm, 4.3 μm, 4.4 μm, 4.5 μm, 4.6 μm, 4.7 μm, 4.8 μm, 4.9 μm, 5.0 μm, or greater than 5.0 μm in width.

Nanochannels may be about 5 nm to about 80 nm in height, about 5 nm to about 8 nm in width, or exactly or about less than 4 nm, 5 nm, 6 nm, 7 nm, 8 nm, 9 nm, 10 nm, 11 nm, 12 nm, 13 nm, 14 nm, 15 nm, 16 nm, 17 nm, 18 nm, 19 nm, 20 nm, 21 nm, 22 nm, 23 nm, 24 nm, 25 nm, 26 nm, 27 nm, 28 nm, 29 nm, 30 nm, 31 nm, 32 nm, 33 nm, 34 nm, 35 nm, 36 nm, 37 nm, 38 nm, 39 nm, 40 nm, 41 nm, 42 nm, 43 nm, 44 nm, 45 nm, 46 nm, 47 nm, 48 nm, 49 nm, 50 nm, 51 nm, 52 nm, 53 nm, 54 nm, 55 nm, 56 nm, 57 nm, 58 nm, 59 nm, 60 nm, 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, 101 nm, 102 nm, 103 nm, 104 nm, 105 nm, 106 nm, 107 nm, 108 nm, 109 nm, 110 nm, 111 nm, 112 nm, 113 nm, 114 nm, 115 nm, 116 nm, 117 nm, 118 nm, 119 nm, 120 nm, 121 nm, 122 nm, 123 nm, 124 nm, 125 nm, 126 nm, 127 nm, 128 nm, 129 nm, 130 nm, 131 nm, 132 nm, 133 nm, 134 nm, 135 nm, 136 nm, 137 nm, 138 nm, 139 nm, 140 nm, 141 nm, 142 nm, 143 nm, 144 nm, 145 nm, 146 nm, 147 nm, 148 nm, 149 nm, 150 nm, 151 nm, 152 nm, 153 nm, 154 nm, 155 nm, 156 nm, 157 nm, 158 nm, 159 nm, 160 nm, 161 nm, 162 nm, 163 nm, 164 nm, 165 nm, 166 nm, 167 nm, 168 nm, 169 nm, 170 nm, 171 nm, 172 nm, 173 nm, 174 nm, 175 nm, 176 nm, 177 nm, 178 nm, 179 nm, 180 nm, 181 nm, 182 nm, 183 nm, 184 nm, 185 nm, 186 nm, 187 nm, 188 nm, 189 nm, 190 nm, 191 nm, 192 nm, 193 nm, 194 nm, 195 nm, 196 nm, 197 nm, 198 nm, 199 nm, 200 nm, or greater than 200 nm in height and width.

### Fabrication

The present disclosure comprises methods for fabricating silicon NWs. It also relates to fabrication of nanochannels and nanowells. The current invention also suggests a method of sampling and manipulating DNA, it also proposes a method of detecting charge contained in the fabricated nanochannel by the included or neighboring NW device, devices or array of NW sensory elements. The length of the NW channel will in one embodiment be longer than a DNA base-pair length, in another embodiment extend beyond a full DNA sequence, in another embodiment it will be comparable in length for long read length DNA sequences, in another embodiment it will facilitate shotgun sequencing, in another embodiment it will be multiple parallel channels.

NWs and nanochannels are typically fabricated using active silicon layers supported on an underlying insulating material. This is typically, but not limited to, Silicon (or polysilicon) on insulator (SOI) wafers where the minimum feature on the active device layer or nanochannel is in one embodiment, less than 500 nm, in another embodiment less than 100 nm, in another embodiment, less than 50 nm, in another embodiment, less than 30 nm, in another embodiment less than 10 nm, in another embodiment less than 5 nm, in another embodiment, less than 2 nm, in another embodiment less than 1 nm.

For NW devices the conductance in one instance may be bulk modified using implantation of various materials to increase the electron doping. In another instance this may be selective to defined NW regions, in another instance this may occur as a single step, in another instance this may be through multiple doping steps. In another instance the conductance may be increased in one region and reduced in another through selective implantation or doping.

Features for the NWs and Nanochannels are defined on the active device surface using and not limited to attachment of pre-defined molds, chemical vapor deposition, physical vapor deposition, oxidation, sputtering, evaporative deposition, photolithographic patterning techniques which may include LTV lithography, interference lithography, e-beam lithography, shadow masking, nanostamping, nanoembossing and nanoink direct writing. Subsequently, unwanted features are either chemically or physically removed to realize or retain the desired feature height and channel width dimensions.

Selective removal of atomic layers can be achieved, targeted, and supplemented, but not limited by, chemical specificity and be inclusive of energetic ion bombardment. One such embodiment includes Focused Ion Beam milling (FIB). Some embodiments comprise gaseous reactive ion etching or plasma etching. Some embodiments are not limited by wet ionic etching and will incorporate a nanofluidic channel across specifically geometrically positioned nanotube, atomically thick layer of graphene or nanowire FET arrays and in some embodiments this may ‘trim’ the nanowires to reduce their dimensions. In some embodiments this my alter the surface to increase their sensitivity. In one embodiment, NW and nanochannel dimensions may be further affected by oxidative and reductive surface chemistries. In some embodiments additional surface layers may be deposited after removal of atomic layers through techniques familiar to those with knowledge in the field. Some embodiments may combine two or more, up to and including all of the above approaches.

Some embodiments have all the NW devices electrically independent of each other within the nanochannel. Some embodiments have multiple NW connected in parallel with each other within the nanochannel.

One embodiment has dielectric (or insulating) material deposited and not limited to Atomic layer deposition, chemical vapor phase deposition, physical vapor deposition, sputtering, molecular beam epitaxy, and Nano dip lithography. Examples of such surface deposited materials are not exclusive to polymer materials, Al2O3, SiN, TiO, SiO2 thermally grown, natural evolution of a native SiO2 layer.

In some embodiments the inclusion of electrically active NWs parallel to the flow of the incoming solution is proposed. In one embodiment the arrangement may be similar to that of a ‘ten-pin-bowling’ pin arrangement and extending in a 1, 2, 3, 4, 5 . . . N arrangement and existing on a single plane. Another will have a Fibonacci incremental arrangement sequence confined within the channel width dimension but existing on the plane of the underlying insulating or dielectric material. Another embodiment has a hexagonal close packed arrangement of NWs existing on the plane of the underlying material. Some embodiments have a mathematically irregular arrangement of NWs. Some embodiments have a random distribution of NWs. Some embodiments have a geometrically regular arrangement of NWs.

Some embodiments have more than one plane of nanochannels isolated from each other. One such realization is an upper and lower channel servicing the top surface of the nanowire and an underlying channel interfacing with the backside of the NW. In this way the functional aspects of the same nanowire can be affected by more than one independent chemistry.

In some embodiments nanochannel nanowire sequencers are fabricated using Graphene transistors as the nanostructure. These can be as sheets flat on the bottom of a nanochannel, or stood up, such that the single atom width of the graphene is perpendicular to the channel allowing for single base resolution. Some embodiments have the graphene transistor or conductor at an angle between the normal and perpendicular axis,

In some embodiments, target DNA sequences can be sequenced in a nanofluidics channel arrayed with sensitive detection nanostructures, like sleepers on a railway track.

The genomic, or other nucleotide polymer molecule sample can be unraveled and elongated into the nanofluidic channel, either in its natural format, or fragmented into fragments>1 kb, or>10 kb, or>1 mb, or>1 gb, or entire chromosomes, from telomere to telomere (T2T Sequencing). In some embodiments, the channel dimensions are such that the DNA, or other polymer molecule, such as RNA, is unable to fold, or form other 3D formations or structures and passes through the channel linearly. Furthermore, the dimensions of the channel are such that the DNA passes within the assay region of the sensitive nanoarray region, thus allowing for each nucleotide within the DNA polymer to cause its unique change in properties in the sensitive nanostructure sensor, thus allowing for sequencing.

In some embodiments an exonuclease enzyme can cleave the terminal nucleotides from trapped (either mechanically, electrically, or other) DNA molecules in the channel. As the cleaved nucleotides pass the sensors, the sensor picks up their unique signature.

In some embodiments, the present invention can be deployed in a handheld device. In further embodiments, this handheld device can sequence a human genome.

In yet further embodiments of this disclosure, the present invention can be incorporated in to a mobile phone. In further embodiments, this mobile phone device can sequence a human genome.

In some embodiments nanochannels are generated using nanoprinting, embossing or direct writing. In other embodiments nanochannels are defined using photolithographic masking techniques including but not limited to contact masking, projection masking, shadow masking, dielectric masking, spacer lithography, electron beam lithography for the microfabrication of nanochannels. Alternately or in combination, nanochannels, such as nanochannels less than 100 nm in depth or in width, may be defined, etched or milled into a predefined nanofabrication structure. This modification may retrospectively create wells or channels consistent with the disclosure herein. Pursuant to this process, additional topographical features or structures may be added, for example to aid in the transport of nucleic acids such as DNA.

In some embodiments, the surface of a suitable substrate is etched using mechanical abrasion. This abrasion may be delivered, for example using a force-controlled cantilever drawn across the surface of the substrate. Mechanical abrasion, milling, troughing or other mechanical abrasion technique may be controlled through the manipulation of an applied tip pressure, angle, tip velocity and tip material. Tip materials consistent with the disclosure herein are silicon, quartz and diamond, although other tip materials are also contemplated.

Additionally or in combination, chemical abrasion may be used to etch a surface. In some embodiments the chemical etching substance is located at the tip of a mechanical etching device as contemplated above (somewhat like that of the ink on a quill or fountain pen in some embodiments), and may be selectively applied at the foci of the tip onto the surface. Chemical substances used herewith may enhance the etching process or may positively affect the transport of the material from the surface and better define channel dimensions, or both enhance the etching and positively affect transport.

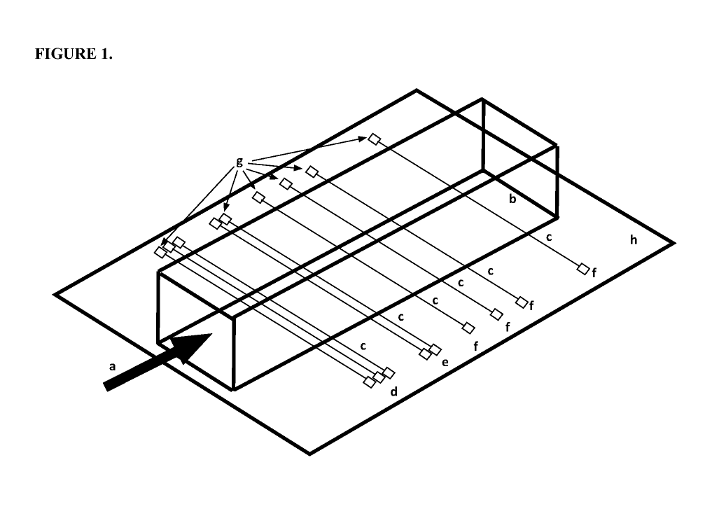

Referring again to the figures, one sees at FIG. 1 a schematic nanochannel nanowire sequencing device of the present disclosure. The elongating single-stranded polynucleotide molecule flows (a) into and through the nanochannel (b). Lining the base, or sides, or top, of the nanochannel are sensitive nanostructure sensors (c). In the illustrated example, the sensors are nanowire FET sensors. These sensitive nanostructure sensors are specifically geometrically spaced such that the system is able to optimally detect the individual bases as they pass, in polymer (DNA or RNA), past them, either individually or as a combined signal deduced and calculated from the signals from a number of nanowires, through their impact on the local electromagnetic environment in the operable vicinity of the nanowires. The nanowires can operate in clusters of 3 (d), 2 (e), singly (f) or in other combinations of any amounts of nanowire clusters. The nanowires are contacted with the electronics via contact pads (g) and the entire device fabricated on a standard silicon chip (h).

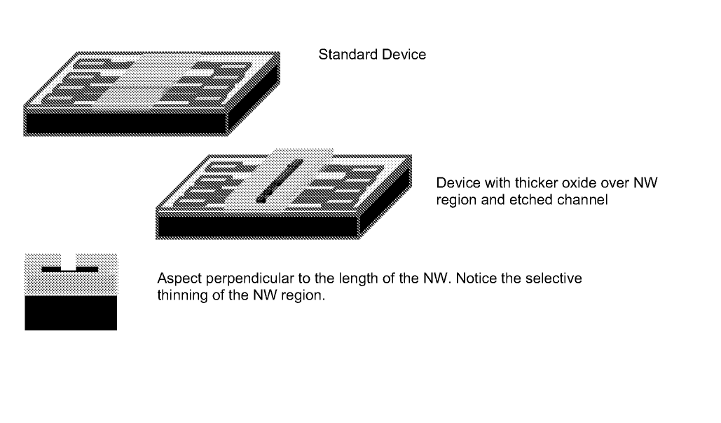

At FIG. 2 one sees multiple views in the manufacture of an embodiment herein. At top is seen a standard device. At middle one sees a nanowell that has been etched into the standard device enhance the sensitivity. At bottom one sees a horizontal view looking down the etched well of the device seen at middle.

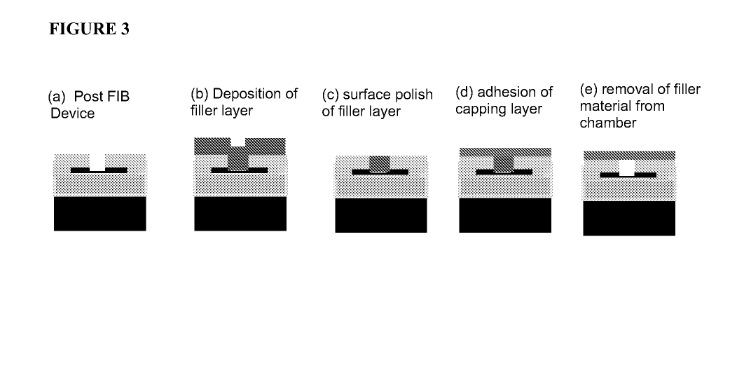

At FIG. 3 one sees a series of steps in the manufacture of a nanochannel as contemplated herein. Following inclusion of the FIB nanochannel along the surface of the device (a), there is an inclusion of bulk material to fill the channel (b) such that it can support and protect the NW region for the capping step and completion of the nanochannel structure. The surface can be polished or etched (c) to remove bulk material outside of the nanochannel track. An adhesion of capping layer is added across the top surface of the device (d). The material in the nanochannel is removed as the last stage in processing of the device, generating a device having a covered, hollowed-out nanochannel (e).

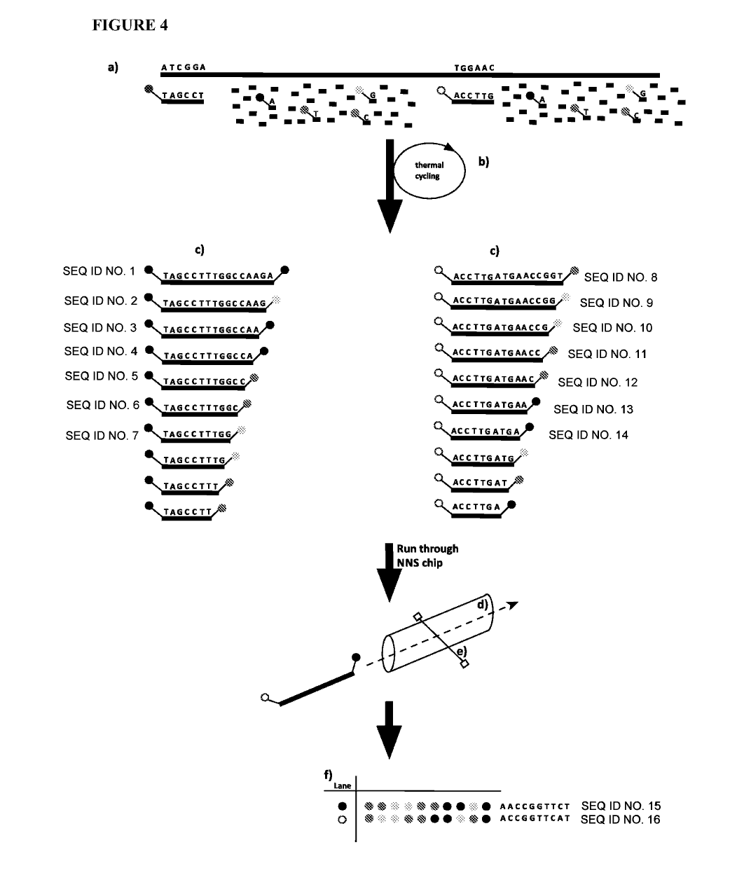

At FIG. 4 one sees Sequencing reaction employing tagged oligonucleotide primer sequence and tagged chain-terminating nucleotides. At view a) Sanger sequencing primers are designed for a template DNA molecule, with multiple primers designed along the length of the region of interest. Each primer will have a unique reporter moiety (reporting based on charge—or size if displacement of buffer is the mode of detection used). The primers and template will be added to the sequencing mix along with dNTPs, with some of the dNTPs in the mix being chain-terminating dNTPs. Each of the chain terminating dNTPs will carry a unique reporter moiety. The concentration of the chain terminating dNTPs will be such that, like Sanger sequencing, different lengths c) of chains will be amplified (either using standard thermal cycling, or isothermally) b). These different lengths will be fed through the nanochannels d), thus contacting each amplified fragment with the arrays of nanowires (only one nanowire is depicted in the image, however, in some embodiments of the device there are hundreds to thousands of nanowires), e). As the first nucleotide (the chain terminating nucleotide) and its reporter moiety passes the sensitive detection nanostructure sensors (in this case a nanowire) it is detected. Next the second reporter moiety, attached to the primer, passes the sensor and is also detected. In some embodiments, it is possible that the chain terminating nucleotide passes through first and then the primer end, without affecting the analysis. As the speed of flow through the nanochannel is known or can calibrate using control DNA fragments of known length, the time between the first reporter detection event and the second reporter detection event provides information of the length of that fragment. The reporter on the primer denotes the location of the start-point on the target DNA molecule and the reporter on the chain terminating nucleotide denotes the base at that particular position, as determined by the length analysis, or calibration.

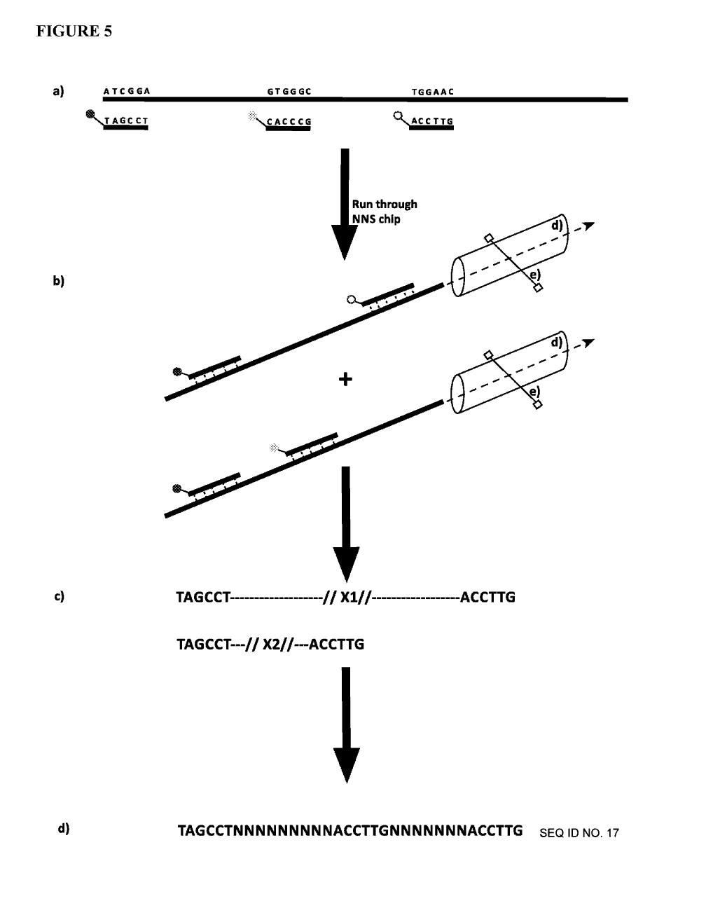

At FIG. 5 one sees an alternate sequence determination consistent with the nanochannel device disclosed herein. The sequence determination method involves a probe-based sequence-detection employing labeled hexamer probes. At (a) all variations of short oligo probes (2, 3, 4, 5, or 6-mers may be used; the figure depicts 6-mers) are synthesized. The probes can be synthesized without reporter moieties or other ligands attached, or each one can carry a different reporter molecule. These probes are added to a solution containing DNA. The solution is heated to melt the DNA and then cooled to allow the probes to hybridize along the length of the ssDNA target molecule. b) The target molecule, or target molecules, with probes attached, are then fed into the nanochannels. The sensitive nanostructure structure (e.g. a nanowire FET) detects the probes, and/or reporter moieties attached to the probes. As the speed of the DNA passing by the sensor, and/or sensors, is known, the positions of the probes can be mapped along the target molecule. As the sequences of the probes are known these can be inferred on the target molecule. Multiple passes of target molecules through the nanochannel sequencers will allow for the full sequence to be computationally built.

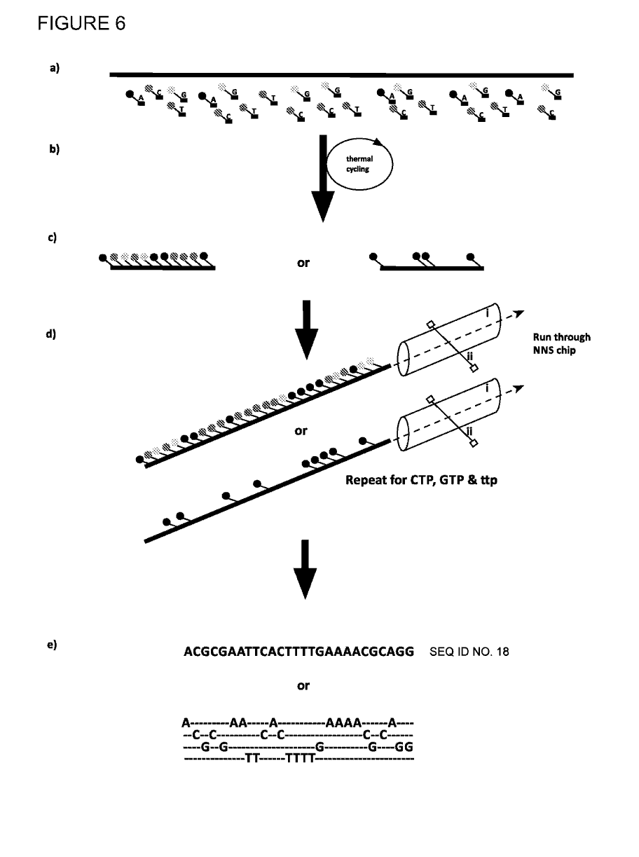

At FIG. 6 one sees an amplification-based sequence detection employing labeled nucleotides. At (a) the target molecule is amplified (b) with dNTPs that carry unique base-specific reporter moieties to generate a complement to the target molecule having labeled nucleic acid bases (c, left). Alternately, four separate reactions with standard nucleotides and one of GTP, CTP, TTP, or ATP with unique reporter moieties attached (c, right). Either alternative will result in amplicons (c) with either every nucleotide along the polymer with a reporter moiety attached (left), or a polymer with one of either GTP, CTP, TTP, or ATP with unique reporter moieties attached (right). At (d) these amplified polymers are then fed through the nanochannel sequencer. At (e) one sees the output for a single pass through a nanochannel At e, top, a product labeled as in c, left, in the case of polymers with all four nucleotides carrying the reporter moiety the sequence of each amplified polymer will be read directly. At e, bottom, in the case of polymers with only GTP, CTP, TTP, or ATP with unique reporter moieties attached, the single bases will be read and spaced due to knowing the speed of the polymer as it passes the sensitive nanostructure sensors (e.g. nanowire FETs) and the full sequences built bioinformatically once all four polymers (representing all of the four nucleotides) have been sequenced.

At FIG. 7 (referring to FIGS. 7A-7G generally) is seen multiple examples of high charge moieties consistent with the NNS detection devices herein.

At FIG. 8 are seen exemplary linker moieties comprising amino acid repeat units that incorporate R groups indicated therein that can carry charge that can affect the FET device. The polypeptide linker, polyglycine in this example, is fused to a charged species comprising one or more of the amino acid residues Aspartic acid, glutamine, serine, threonine, tyrosoine, alanine, and glycine that may comprise a charged species. These species may also be able to act as a chelating group to bind other species such as magnetic or paramagnetic ions or particles.

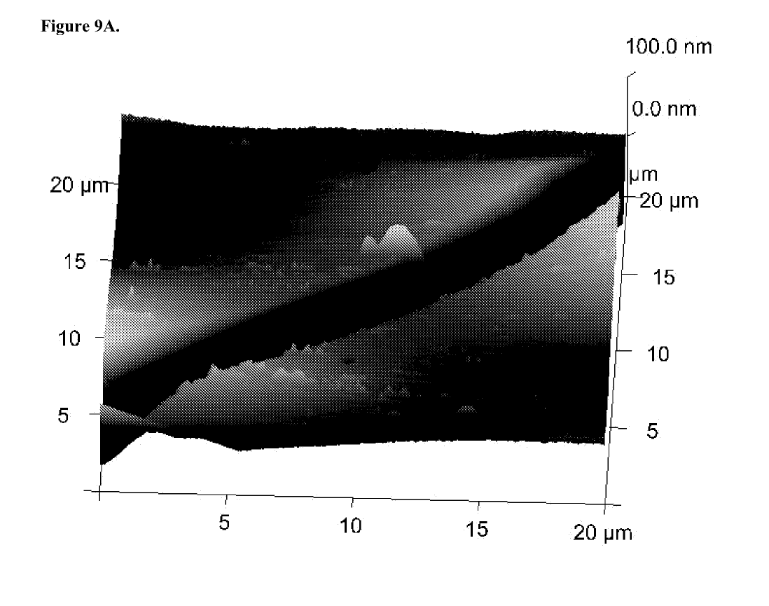

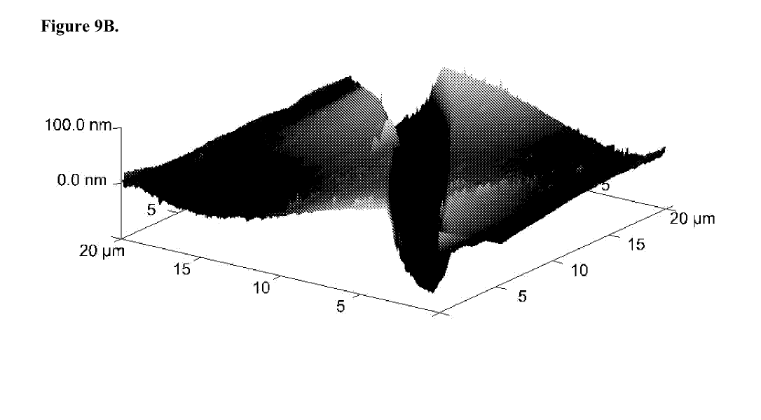

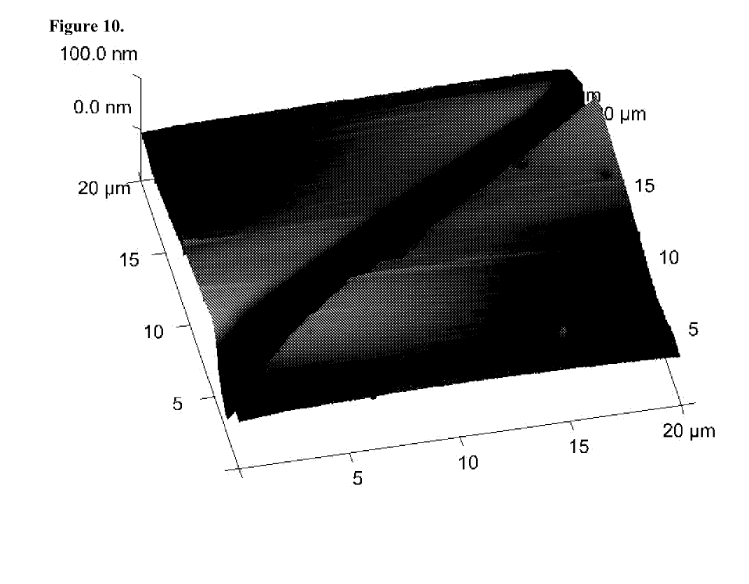

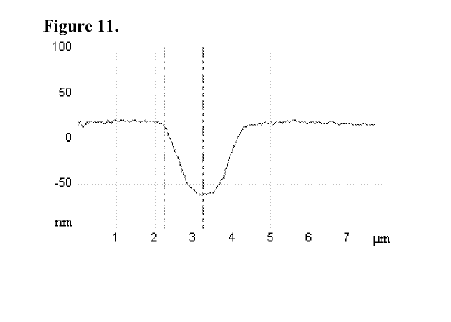

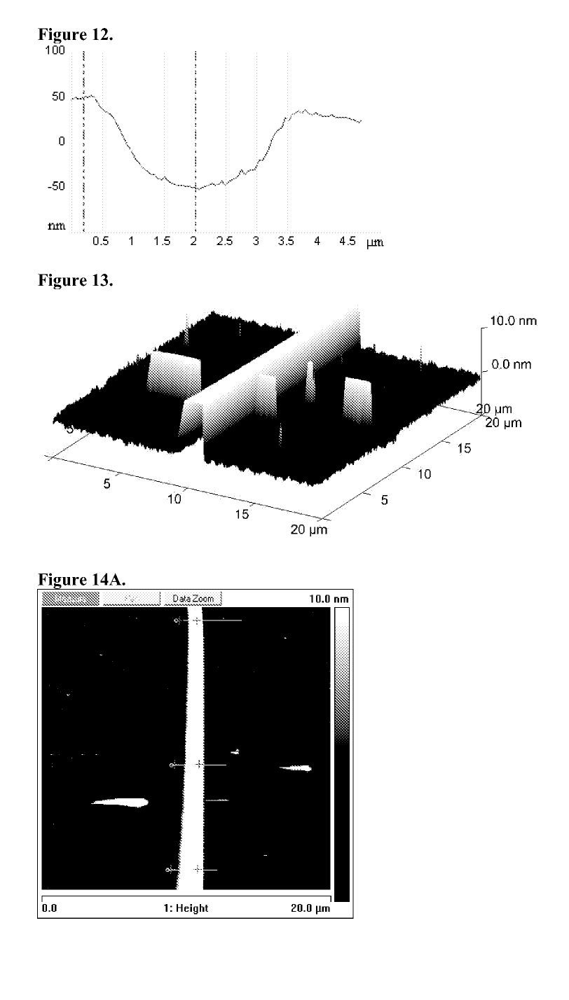

At FIGS. 9A through 14B, one sees images and measurements of exemplary nanochannels consistent with the devices and methods disclosed herein. Nanochannel height and width are consistently, uniformly reproduced.

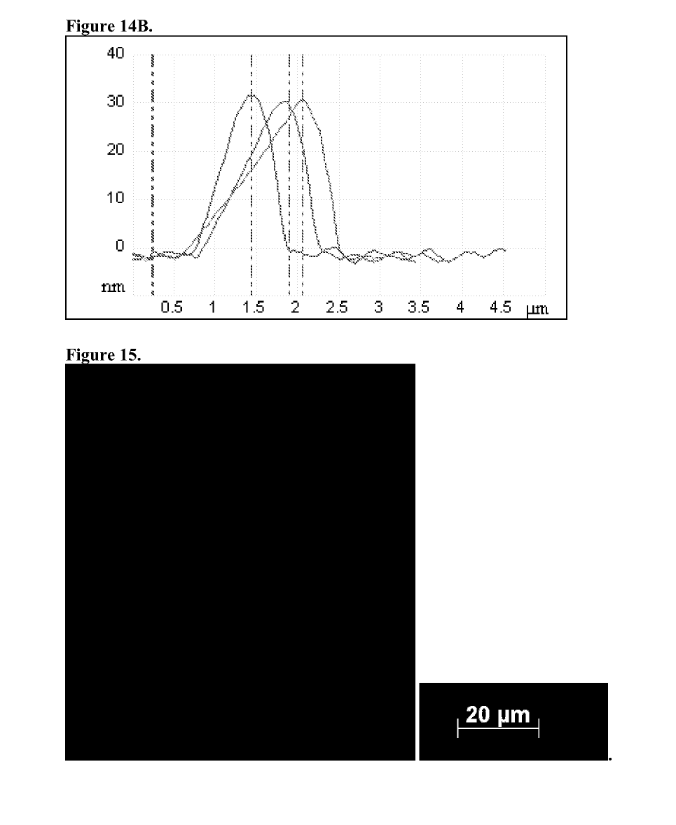

At FIG. 15 one sees a Cy3-labeled DNA sample accumulating in a chamber at the end of a nanochannel. This figure demonstrates that nucleic acids can be drawn through nanochannels consistent with the devices and methods disclosed herein.

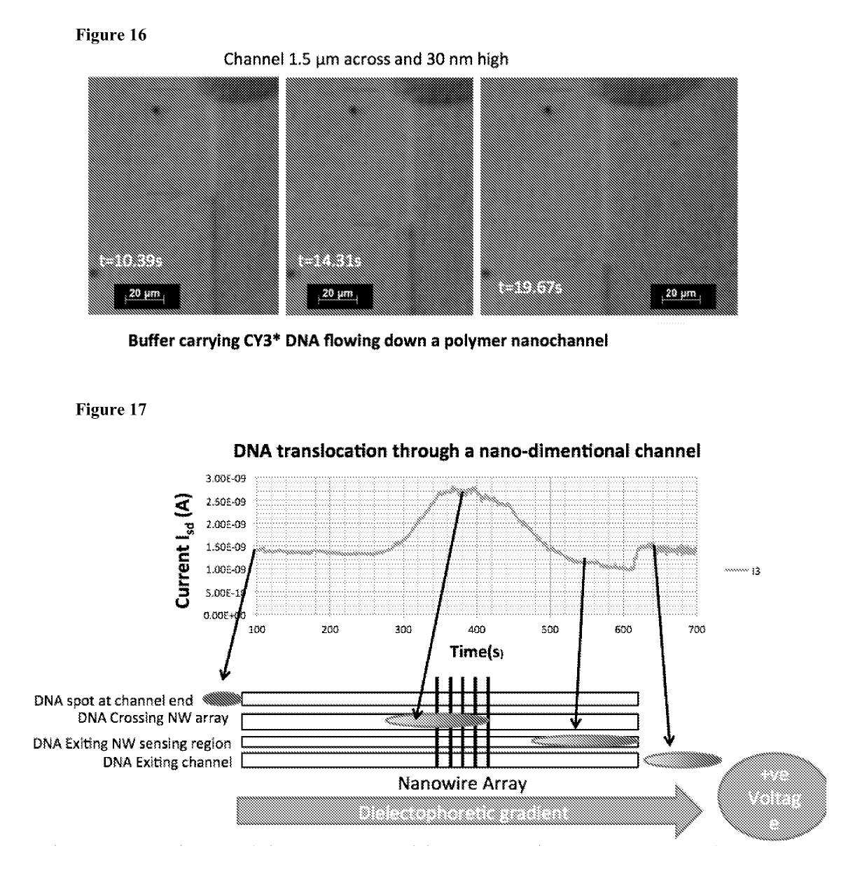

At FIG. 16 a template was engineered through the printing of a topography continuous structure of linewidth 1.5 um, height 50 nm and length 3 mm on a silicon wafer. A liquid polymer was degassed and applied to the surface and subsequently cured. Upon removal of the polymer the channel was hydrophilisied. As can be seen in the Figure, the channel directs solution containing DNA in a controlled manner at approximately Sum per second. The progression of a solution through the channel is seen through comparison of the left, center and right panels of FIG. 16, which represent a time-course of the progression of a sample comprising a buffer carrying CY3* DNA through the nanochannel.

At FIG. 17 DNA (10 um) was injected to one end of the nano-dimensional channel positioned to cross a NW array. Sampling rate was 10 Hz owing to limitations of the hardware. Additional to the concentration gradient effects, a dielectrophoretic gradient was established to introduce additional mobility to the DNA in the channel. Passage of the DNA across the nanowire array was observed through its effect on the current Isd (A), at 350-450 s, depicted a ttop. At middle, one sees a schematic of polynucleic acid location in a nanochannel as indicated at the left of the middle schematic. Arrows correspond each middle schematic to a measured current. At bottom is indicated in the direction of the electrophoretic gradient.

### EXAMPLES

The followings are some illustrative and non-limiting examples of some embodiments of the present disclosure.

### Example 1

### CMOS Synthesis

To develop nanowells or nanochannels a thicker layer, typically but not limited to 35 nm, of Al2O3 (or SiO2) is deposited on the active NW region. Some designs are fabricated to have 35 nm tall NWs on the underlying oxide. A 3 nm AlO3 dielectric layer is blanket deposited resulting in the inter-nanowire region (valley) of the device having a 3 nm AlO3 layer over oxide and the 35 nm NW combining to a height of 38 nm.

In an alteration to this fabrication methodology, a secondary 35 nm AlO3 (or SiO2) is deposited, giving a valley height of 38 nm AlO3 over oxide and approximately 70 nm height inclusive of AlO3 and NW. One non-limiting embodiment utilizes a Focused Ion Beam (FIB) to remove 20 nm of material in the valley regions of the channel in the AlO3 and 50 nm above the NW to planarize a channel. This may have the effect of including a 20 nm fluidic channel in the AlO3 and thinning the NW to 20 nm (removing 15 nm of Si and 35 nm AlO3 from the surface). Thinning the NW enhances the sensitivity in two ways. Firstly, a focused E-field will develop across the ‘pinched’ region of the NW; and secondly a reduction in the local conductance at the channel crossing point will occur.

When the dimensions of the NW and nanochannel devices have been achieved, conductive properties of the devices may be enhanced at edges for connection to external circuitry.

### Example 2

### NextGen Sanger Sequencing (NSS)

Sanger sequencing primers are designed for a template DNA molecule, with multiple primers designed along the length of the region of interest. Each primer has a unique reporter moiety (reporting based on charge—or size if displacement of buffer is the mode of detection used). The primers and template are added to the sequencing mix along with dNTPs, with some of the dNTPs in the mix being chain-terminating dNTPs. Each of the four chain terminating dNTPs carry a unique reporter moiety. The concentration of the chain terminating dNTPs are such that, like Sanger sequencing, different lengths (FIG. 4, c) of chains are amplified (either using standard thermal cycling, or isothermally) (FIG. 4, b). These different lengths are fed through the nanochannels (FIG. 4, d), thus contacting each amplified fragment with the arrays of nanowires (only one nanowire is depicted in the image, however, in the final device there will be hundreds to thousands of nanowires), (FIG. 4, e). As the first nucleotide (the chain terminating nucleotide) and its reporter moiety passes the sensitive detection nanostructure sensors (in this case a nanowire) it is detected. Next the second reporter moiety, attached to the primer, passes the sensor and is also detected. Note, it is possible that the chain terminating nucleotide passes through first and then the primer end, it makes no difference to the analysis. As the speed of flow through the nanochannel is known (or can be calibrated using control DNA fragments of known length) the time between the first reporter detection event and the second reporter detection event provides information of the length of that fragment. The reporter on the primer denotes the location of the start-point on the target DNA molecule and the reporter on the chain terminating nucleotide denotes the base at that particular position, as determined by the length analysis, or calibration.

### Example 3

### NextGen Probe Based Sequencing (NPS)

All variations of short oligo probes (2, 3, 4, 5, or 6 mers) are synthesized. The probes are optionally synthesized without reporter moieties or other ligands attached, or each one can carry a different reporter molecule. These probes are added to a solution containing DNA. The solution is heated to melt the DNA and then cooled to allow the probes to hybridize along the length of the ssDNA target molecule. (FIG. 5, b) The target molecule, or target molecules, with probes attached, are then fed into the nanochannels. The sensitive nanostructure structure (e.g. a nanowire FET) detects the probes, and/or reporter moieties attached to the probes. As the speed of the DNA passing by the sensor, and/or sensors, is known, the positions of the probes can be mapped along the target molecule. As the sequences of the probes are known these can be inferred on the target molecule. Multiple passes of target molecules through the nanochannel sequencers will allow for the full sequence to be computationally built.

### Example 4

### NextGen Tagged Nucleotide Sequencing (NTN)

The target molecule is amplified (FIG. 6, b) with dNTPs that carry unique reporter moiety. OR four separate reactions with standard nucleotides and one of GTP, CTP, TTP, or ATP with unique reporter moieties attached. This will result in amplicons (FIG. 6, c) with either every nucleotide along the polymer with a reporter moiety attached, or a polymer with one of GTP, CTP, TTP, or ATP with unique reporter moieties attached. (FIG. 6, d) these amplified polymers are then fed through the nanochannel sequencer. (FIG. 6, e) in the case of polymers with all four nucleotides carrying the reporter moiety the sequence of each amplified polymer will be read directly. In the case of polymers with only GTP, CTP, TTP, or ATP with unique reporter moieties attached, the single bases will be read and spaced due to knowing the speed of the polymer as it passes the sensitive nanostructure sensors (e.g. nanowire FETs) and the full sequences built bioinformatically once all four polymers (representing all of the four nucleotides) have been sequenced.

### Example 5

### Polynucleic Acid Drawn Through a Nanochannel

The DNA molecules were labeled with Cy3 and drawn through nanochannels consistent with the disclosure herein. Red fluorescence accumulates in a pool at the terminus of a nanochannel, demonstrating that nucleic acids can be drawn through nanochannels as contemplated herein.

### Example 6

### Fabrication of a Graphene NNS Device

Nanochannel nanowire sequencers are fabricated initially by depositing a grapheme sheet on to a surface and then performing layer deposition, either physically, chemically or atomically, of a material such as, but not limited to silicon oxide, silicon nitride, polymers, kapton and inclusive chemistries, SU8, or other photoresist, etc., until one has built of a sufficient height with a height divisible by 3.4 angstroms (the base to base distance in DNA). Then a second sheet of grapheme is deposited, grown or otherwise manipulated on top. Further layer deposition (inclusive but not limited to the afore mentioned techniques) is performed and further grapheme layers established until there are between 1 and 1,000 layers of Graphene. These layers are optionally then diced and turned 90 degrees. Optionally, these layers may be used as defined by the fabricating process. A nanochannel is formed in layers, perpendicular to the grapheme and the graphene stack or column is then coupled onto a CMOS chip containing a number of discrete (or otherwise electrically useful arrangement of) source and drain electrodes, such that the graphene sheets connect the electrodes and form nanostructure sensors.

1. A device for sequencing a polynucleic acid molecule, the device comprising:
a nanochannel having a height and width of less than 100 nm; and
a nanostructure sensor having a sensitive assay region within said nanochannel such that a perturbation resulting from an individual base of a polynucleic acid passing through said sensitive assay region results in a specific signal being generated by said sensor.

2. (canceled)

3. (canceled)

4. The device of claim 1, wherein said nanochannel is bounded by walls comprising at least one of Al₂O₃, SiN, Si, grapheme, polymetric materials, photoresist and SiO₂.

5. The device of claim 1, wherein said nanochannel comprises a capping layer.

6. (canceled)

7. The device of claim 1, wherein said nanostructure sensor comprises one or more selected from the group consisting of a nanowire, a carbon nanotube, graphene, and an FET device.

8. (canceled)

9. (canceled)

10. (canceled)

11. The device of claim 1, wherein said nanostructure sensor detects one or more selected from the group consisting of electrical charge, buffer solution potential, fluorescence, buffer displacement, and heat.

12. The device of claim 1, wherein said nanostructure detects a high-charge moiety.

13. (canceled)

14. (canceled)

15. (canceled)

16. (canceled)

17. (canceled)

18. (canceled)

19. The device of claim 1, further comprising an array of nanostructure sensors positioned within said nanochannel such that individual bases of a polynucleotide molecule passing by said sensors are detected.

20. (canceled)

21. (canceled)

22. (canceled)

23. (canceled)

24. The device of claim 1, wherein said nanochannel can hold a solution.

25. (canceled)

26. The device of claim 24, wherein said solution conducts an electric current that draws the polynucleic acid into or through said nanochannel.

27. (canceled)

28. (canceled)

29. The device of claim 1, wherein said device is sized and configured to be hand-held.

30. A method of sequencing a polynucleic acid molecule, the method comprising:
drawing said polynucleic acid molecule;
past a sensitive assay region of a nanostructure sensor; and
measuring a perturbation in said sensitive assay region,
wherein said perturbation corresponds to an individual base of said polynucleic acid molecule.

31. The method of claim 30, wherein said perturbation is comprises one or more selected from the group consisting of an electric charge in said sensitive assay region, a volume displacement in said sensitive assay region, a solution potential in said sensitive assay region, fluorescence in said sensitive assay region, and heat in said sensitive assay region.

32. (canceled)

33. (canceled)

34. (canceled)

35. (canceled)

36. (canceled)

37. (canceled)

38. The method of claim 30, wherein drawing said polynucleic acid molecule past said sensitive assay region comprises running a current through a solution comprising said polynucleic acid molecule.

39. The method of claim 30, wherein drawing said polynucleic acid molecule past said sensitive assay region comprises establishing a flow of a solution comprising said polynucleic acid molecule past said sensitive assay region.

40. (canceled)

41. (canceled)

42. (canceled)

43. (canceled)

44. (canceled)

45. (canceled)

46. (canceled)

47. (canceled)

48. A method of sequencing a target polynucleotide, comprising:
providing within an assay region an array of sensitive detection nanostructure sensors each of which generates a signal related to a property of a nucleotide that flows past the array within the assay region, wherein the assay region comprises a nanofluidics channel;
elongating said target polynucleotide through the nanofluidics channel, such that the target polynucleotide passes within an operable field of at least one sensitive nanostructure sensor; and
detecting within the assay region a change in the signal that is characteristic of at least one nucleotide base in said target polynucleotide.

49. (canceled)

50. The method of claim 48, further comprising detecting first and second signals related to first and second nucleotide bases, respectively, wherein a flow rate of the elongated target polynucleic acid in the assay region is known, such that a length between the first and second nucleotide bases may be determined.

51. (canceled)

52. (canceled)

53. (canceled)

54. The method of claim 48, wherein the property comprises one or more selected from the group consisting of an electrical charge, fluorescence, conductance, volume, and heat.

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. A microfluidics cassette, comprising:
a sample reception element for introducing a biological sample comprising the a target polynucleotide into the cassette;
a lysis chamber for disrupting the biological sample to release a soluble fraction comprising nucleic acids and other molecules;
a nucleic acid separation chamber for separating the nucleic acids from the other molecules in the soluble fraction;
an amplification chamber for amplifying the target polynucleotide;
an assay region comprising an array of sensitive detection nanostructures each of which generates a signal in response to a change a property of the nanostructures, wherein the assay region is configured to allow an interaction between the nucleotide bases of the target polynucleotide and the nanostructures; and
a conducting element for conducting the signal to a detector.

78. (canceled)

79. (canceled)

80. (canceled)

81. (canceled)

82. (canceled)

83. (canceled)

84. (canceled)

85. (canceled)

86. (canceled)

87. (canceled)

88. (canceled)

89. (canceled)

90. (canceled)

91. (canceled)

92. (canceled)

93. (canceled)

94. (canceled)

95. (canceled)


SEQUENCE LISTING (18 sequences)

SEQ ID NO: 1 - DNA, 16 bp, Artificial sequence
  Synthetic nucleotide
tagcctttggccaaga

SEQ ID NO: 2 - DNA, 15 bp, Artificial sequence
  Synthetic nucleotide
tagcctttggccaag

SEQ ID NO: 3 - DNA, 14 bp, Artificial sequence
  Synthetic nucleotide
tagcctttggccaa

SEQ ID NO: 4 - DNA, 13 bp, Artificial sequence
  Synthetic nucleotide
tagcctttggcca

SEQ ID NO: 5 - DNA, 12 bp, Artificial sequence
  Synthetic nucleotide
tagcctttggcc

SEQ ID NO: 6 - DNA, 11 bp, Artificial sequence
  Synthetic nucleotide
tagcctttggc

SEQ ID NO: 7 - DNA, 10 bp, Artificial sequence
  Synthetic nucleotide
tagcctttgg

SEQ ID NO: 8 - DNA, 16 bp, Artificial sequence
  Synthetic nucleotide
accttgatgaaccggt

SEQ ID NO: 9 - DNA, 15 bp, Artificial sequence
  Synthetic nucleotide
accttgatgaaccgg

SEQ ID NO: 10 - DNA, 14 bp, Artificial sequence
  Synthetic nucleotide
accttgatgaaccg

SEQ ID NO: 11 - DNA, 13 bp, Artificial sequence
  Synthetic nucleotide
accttgatgaacc

SEQ ID NO: 12 - DNA, 12 bp, Artificial sequence
  Synthetic nucleotide
accttgatgaac

SEQ ID NO: 13 - DNA, 11 bp, Artificial sequence
  Synthetic nucleotide
accttgatgaa

SEQ ID NO: 14 - DNA, 10 bp, Artificial sequence
  Synthetic nucleotide
accttgatga

SEQ ID NO: 15 - DNA, 10 bp, Artificial sequence
  Synthetic nucleotide
aaccggttct

SEQ ID NO: 16 - DNA, 10 bp, Artificial sequence
  Synthetic nucleotide
accggttcat

SEQ ID NO: 17 - DNA, 34 bp, Artificial sequence
  Synthetic nucleotide
tagcctnnnnnnnnnaccttgnnnnnnnaccttg

SEQ ID NO: 18 - DNA, 27 bp, Artificial sequence
  Synthetic nucleotide
acgcgaattcacttttgaaaacgcagg